(19) United States
(12) Patent Application Publication
(10) Pub. No.: US 2026/0224524 A1
(43) Pub. Date: Aug. 6, 2026

(54) A PHARMACEUTICAL COMPOSITION TARGETING MUTANT P53 AND HER2

(71) Applicant: DR DOZO LABORATORIES, MOHALI, Punjab (IN)

(72) Inventor: Gurdial Singh ARORA, Mohali, Punjab (IN)

(21) Appl. No.: 19/151,638

(22) PCT Filed: Feb. 12, 2024
(86) PCT No.: PCT/IN2024/050138

§ 371 Date: Jul. 29, 2025

(30) Foreign Application Priority Data
Feb. 13, 2023 (IN) 202311009420

(51) Int. Cl. A61K 31/366; A61K 31/351; A61K 31/404; A61K 36/8998; A61K 45/06; A61P 35/00; C07D 309/38; C07D 311/76

(52) U.S. Cl. CPC A61K 31/366; A61K 31/351; A61K 31/404; A61K 36/8998; A61K 45/06; A61P 35/00; C07D 309/38; C07D 311/76; A61K 2236/55

(57) ABSTRACT

The present invention relates to a pharmaceutical composition having anticancer activity. More particularly, the present invention relates to a composition and method for treatment of cancer. Also, the present invention relates to a pharmaceutical composition which targets P53 and HER2.

### FIELD OF THE INVENTION

The present invention relates to a composition having anticancer activity. More particularly, the present invention relates to a composition and method for treatment of cancer.

### BACKGROUND OF THE INVENTION

Cancer is such a disease, which at present seems to be incurable. Moreover, present day chemotherapies given to cancer patients are very-very expensive and beyond common man's reach due to toxic and other side effects of the chemotherapy system (which kills living cells also). The patients lose weight, appetite and are subjected to lot of physical as well as mental harassment. All over the world, scientists are working hard, and a lot of money and efforts are put in to find a cure for the cancer.

Mother Nature stands as an unrestrained and infinite reserve for the advancement of new effective drugs and therapies (R. Nabi et al., 2019). In recent years, the medicinal herbs have gained essential importance in treating diseases, globally. Since the former age, biological and natural compounds have played a significant role in the treatment and prevention of various diseases, forming the backbone of the conventional system of medicine and treatment.

The use of natural compounds as a basis for the development of new drugs or as drugs has been widely adopted as a complementary or alternative option in the oncology field. Therefore, every year, several novel cytotoxic compounds are isolated from plants and constitute new possibilities to fight cancer.

The natural compounds as a drug for the treating cancer come with benefits of reduced adverse effects and the capacity to impact on multiple signalling pathways involved in the carcinogenesis process.

Of course, nature is great, it has solution to all problems, and it is an object of the present invention to provide composition which act as an anticancer with no side effects.

Thus, the present study focuses on the anticancer potential of compounds isolated from barley extract. In this study, isolation of compounds as potential anticancer agents from barley extract, including their structural characterization and ability as anticancer also being investigated.

### OBJECTS OF THE PRESENT INVENTION

The main objective of the present invention is to provide a pharmaceutical composition (SACC) comprising the major compounds of formula (I), formula (II), formula (III), and formula (IV) obtained from Barley extract.

Another object of the present invention is to provide the compounds isolated from the barley extract.

Another object of the present invention is to provide a HPLC method for the detection of all the compounds present in the barley extract.

Another object of the present invention is to provide a method for the isolation of compounds from barley extract.

Another object of the present invention is to provide ESI-MS analysis of the compound and determining the structure of some of the compounds isolated from the barley extract using NMR and IR spectroscopy.

Another object of the present invention is to provide a pharmaceutical composition comprising the compounds of the present invention for treatment of cancer.

Another object of the present invention is to provide a method of preventing or treating a cancer in a subject in need thereof comprising administering a therapeutically effective amount of a pharmaceutical composition comprising the compounds of formula (I), formula (II), formula (III), and formula (IV).

Another object of the present invention is to provide a use of a pharmaceutical composition comprising the compounds of formula (I), formula (II), formula (III), and formula (IV) for the manufacture of a medicament for treating cancer.

Another object of the present invention is to provide a use of a pharmaceutical composition comprising the compounds of formula (I), formula (II), formula (III), and formula (IV) for treating cancer.

Another object of the present invention is to provide a use of a compound of formula (I), formula (II), formula (III), and formula (IV) for the manufacture of a medicament for treating cancer.

Another object of the present invention is to provide a pharmaceutical composition comprising a compound of formula (I), formula (II), formula (III), and formula (IV) for use as a medicament for treatment of prostate cancer, triple negative breast cancer, invasive cancers, lung cancers, sarcoma, serous carcinoma, pancreatic cancer, and HER2 positive breast cancer, in a subject.

### SUMMARY OF THE PRESENT INVENTION

The present disclosure provides a pharmaceutical composition comprising at least one compound selected from the following formulae obtained from the barley extract.
a)

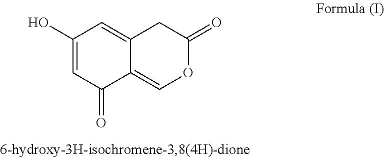 [SMILES: O=C1CC2=CC(O)=CC(=O)C2=CO1]

b)

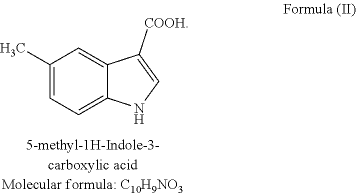

c)

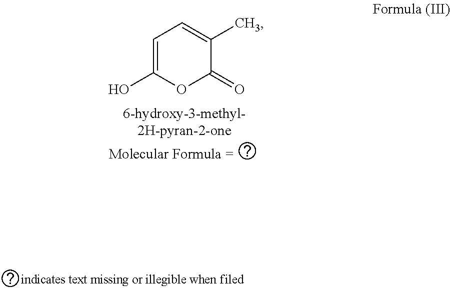

d)

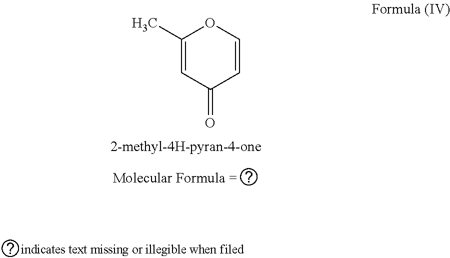

or a combination thereof.

The present disclosure provides a HPLC method for the detection of the compounds of formula (I), (II), (III) and (IV) present in the barley extract.

The present disclosure provides a method for the isolation of said compounds from barley extract.

The present disclosure provides ESI-MS analysis of the compound and determining the structure of compound isolated from the barley extract using NMR and IR spectroscopy.

The present disclosure provides a pharmaceutical composition comprising the compounds of formula (I), formula (II), formula (III), and formula (IV) obtained from Barley extract for (i) inducing apoptosis and inhibiting the growth of pancreatic cancer cells irrespective of mutant p53, (ii) inhibiting the expression of HER-2 (iii) inducing the expression of pro-apoptotic protein Bax and inhibiting anti-apoptotic protein Bcl2, (iv) inducing the PARP protein cleavage, (v) for treatment of prostate cancer, Triple negative breast cancer (TNBC), invasive cancers, lung cancers, sarcoma, serous carcinoma, pancreatic cancer, and HER2 positive breast cancer, in a subject.

The present disclosure provides use of a pharmaceutical composition comprising barley extract as an active ingredient in the manufacture of a medicament for (i) inducing apoptosis and inhibiting the growth of pancreatic cancer cells irrespective of mutant p53, (ii) inhibiting the expression of HER-2 (iii) inducing the expression of pro-apoptotic protein Bax and inhibiting anti-apoptotic protein Bcl2, (iv) inducing the PARP protein cleavage, in a subject, (v) for treatment of prostate cancer, triple negative breast cancer, invasive cancers, lung cancers, sarcoma, serous carcinoma, pancreatic cancer, and HER2 positive breast cancer.

The present disclosure provides a method of inducing apoptosis and inhibiting the growth of pancreatic cancer cells irrespective of mutant p53, (ii) inhibiting the expression of HER-2 (iii) inducing the expression of pro-apoptotic protein Bax and inhibiting anti-apoptotic protein Bcl2, (iv) inducing the PARP protein cleavage, (v) for treatment of prostate cancer, triple negative breast cancer, invasive cancers, lung cancers, sarcoma, serous carcinoma, pancreatic cancer, and HER2 positive breast cancer, in a subject.

The present disclosure provides a pharmaceutical composition for use as a medicament in a subject for treating cancer.

The present disclosure provides a use of the pharmaceutical composition for treating cancer.

The present disclosure provides the pharmaceutical composition for the treatment of pancreatic cancer.

The present disclosure provides the pharmaceutical composition comprising the compounds of the compounds of formula (I), formula (II), formula (III), and formula (IV) obtained from Barley extract for inhibition and treatment of prostate, triple negative breast cancer, invasive cancers, lung cancers, sarcoma, serous carcinoma, pancreatic cancer, and HER2 positive breast cancer.

### BRIEF DESCRIPTION OF DRAWINGS

In order that the disclosure may be readily understood and put into practical effect, reference will now be made to exemplary embodiments as illustrated with reference to the accompanying figures. The figures together with a detailed description below, are incorporated in and form part of the specification, and serve to further illustrate the embodiments and explain various principles and advantages, in accordance with the present disclosure where in:

FIG. 1. Illustrates the HPLC chromatograms of barley extract (crude. B. No: KWO 037) at different wavelengths of UV (in nm).

FIG. 2. Illustrates a typical Preparative HPLC chromatogram.

FIG. 3. Illustrates a typical max plot (200-400 nm) HPLC chromatogram and represents Peak 2.

FIG. 4. Illustrates a typical HPLC chromatogram at 215 nm and represents Peak 2.

FIG. 5. Illustrates IR Spectrum of Peak 2.

FIG. 6. Illustrates ESI mass spectrum of Peak 2.

FIG. 7. Illustrates 1H NMR spectrum of Peak 2.

FIG. 8. Illustrates a typical max plot (200-400 nm) HPLC chromatogram and represents Peak 4.

FIG. 9. Illustrates a typical HPLC chromatogram at 274 nm and represents Peak 4.

FIG. 10. Illustrates IR Spectrum of peak 4.

FIG. 11. Illustrates ESI mass spectrum of Peak 4.

FIG. 12. Illustrates 1H NMR spectrum of Peak 4.

FIG. 13. Illustrates a typical max plot (200-400 nm) HPLC chromatogram and represents Peak 5.

FIG. 14. illustrates typical HPLC chromatogram at 291 nm and represents Peak 5.

FIG. 15. Illustrates IR Spectrum of Peak 5.

FIG. 16. Illustrates ESI mass spectrum of Peak 5.

FIG. 17. Illustrates 1H NMR spectrum of Peak 5.

FIG. 18. Illustrates the Histogram for ( 1/30 Final Dilution SACC) for Kinase reactions.

FIG. 19. Illustrates the Histogram for ( 1/120 Final Dilution SACC) for Kinase reactions.

FIG. 20. Illustrates full Inhibitor Titrations for IC₅₀ Determination.

FIG. 21. Graph showing cell viability after the treatment of Test Sample against A549 cell line.

FIG. 22. Graph showing cell viability after the treatment of standard (Doxorubicin) against A549 cell line.

FIG. 23. Graph showing cell inhibition after the treatment of test sample in MTT Assay against A549 cells.

FIG. 24. Graph representing the inhibitory activity and IC₅₀ value of test sample in MTT Assay against A549 cells.

FIG. 25. Graph showing cell inhibition after the treatment of standard (Doxorubicin) in MTT Assay against A549 cells.

FIG. 26. Graph representing the inhibitory activity and IC₅₀ value of standard (Doxorubicin) in MTT Assay against A549 cells.

FIG. 27 A. Representative line graph illustrating tumour volume at indicated time point of control and SACC administered groups. Value in the graphs is mean of n=8 mice in control and n=9 mice in SACC treated groups. SACC was administered 5 days/week for consecutively 6 weeks.

FIG. 27 B. Representative bar graph illustrating tumour weight of control and SACC groups mice at 9 weeks. Values in the graph are mean of n=8 and n=9 mice of control and SACC treatment groups respectively.

FIG. 28. Images of 12-week study in control and SACC treated mice.

FIG. 29. Represents the line graph for Control group mice AsPC1 cells Xenograft Tumours.

FIG. 30. Represents the line graph for SACC group mice AsPC1 cells Xenograft Tumours.

FIG. 31 A. Represents the images of control and SACC treated mice bearing xenograft tumours. Lower panel pictures indicate excised xenograft tumours of control and SACC treated mice.

FIG. 31 B. Shows tumour volume of xenograft tumours in control and SACC treatment at indicated week where blue line indicates control and red line indicates SACC. FIG. 31 C. Depicts excised xenograft tumours weight of control and SACC treated mice at week 5.

FIG. 32. Histopathology results of Pan-CSCs derived xenograft tumours of control and SACC treated mice.

FIG. 33. Effect of SACC on expression of transcription factors GLI-1 and chemokine receptor CXCR4. Representative Immunohistochemistry images of GLi-1 and CXCR4 expression of control and SACC xenograft tumours.

FIG. 34. Effect of SACC on pancreatic cancer cells viability and apoptosis:
A. Effect of SACC on cell growth of AsPc1 cells as determined by MTT assay.
B. Effect of control and SACC on apoptosis induction in MiaPaCa-2 cells as assessed by flow cytometry.

FIG. 35. Effect of SACC on the expression of HER-2/p53 and apoptotic proteins in pancreatic cancer cells.

FIG. 36. Demonstrates the percentage area of peaks in HPLC chromatogram.

FIG. 37 Effect of SA1 (peak 5) and SA2 (peak 4) in human breast cancer cell line MDA-MB-231 presented by growth curve as compared to Control (Adriamycin).

FIG. 38. Effect of SACC in human breast cancer cell line MDA-MB-231 presented by growth curve as compared to Control (Adriamycin).

FIG. 39 Effect of SA1 (peak 5) and SA2 (peak 4) in human lung cancer cell line A-549 presented by growth curve as compared to Control (Adriamycin).

FIG. 40. Effect of SACC in human lung cancer cell line A-549 presented by growth curve as compared to Control (Adriamycin).

FIG. 41. Effect of SA1 (peak 5) and SA2 (peak 4) in human pancreatic cancer cell line Mia-Pa-Ca-2 presented by growth curve as compared to Control (Adriamycin).

FIG. 42. Effect of SACC in human pancreatic cancer cell line Mia-Pa-Ca-2 presented by growth curve as compared to Control (Adriamycin).

FIG. 43. Represents a graph for Relative Tumour Volume (RTV) in human tumour xenograft model MDA-MB-231 for Groups A-D.

FIG. 44. Represents a graph for T/C values from RTV data in human tumour xenograft model MDA-MB-231 for Groups A-D.

FIG. 45. Represents a graph for percentage of survival rate in human tumour xenograft model MDA-MB-231 for Groups A-D.

FIG. 46 Represents a graph for average animal body weight in human tumour xenograft model MDA-MB-231 for Groups A-D.

FIG. 47. Represents a graph for tumour volume in human tumour xenograft model MDA-MB-231 for Group A.

FIG. 48. Represents a graph for tumour volume in human tumour xenograft model MDA-MB-231 for Group B.

FIG. 49. Represents a graph for tumour volume in human tumour xenograft model MDA-MB-231 for Group C.

FIG. 50. Represents a graph for tumour volume in human tumour xenograft model MDA-MB-231 for Group D.

### DETAILED DESCRIPTION OF THE INVENTION

At the very outset of the detailed description, it may be understood that the ensuing description only illustrates a particular form of this invention. However, such a particular form is only exemplary embodiment, and without intending to imply any limitation on the scope of this invention. Accordingly, the description is to be understood as an exemplary embodiment and teaching of invention and not intended to be taken restrictively.

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

As used herein, the term ‘compound(s)’ comprises the compounds disclosed in the present invention.

As used herein the SACC refers to the composition comprising compounds of formula (I), formula (II), formula (III), and formula (IV) obtained from Barley extract.

As used herein, the term “comprises” or “comprising” is generally used in the sense of include, that is to say permitting the presence of one or more features or components.

As used herein, the terms “optional” or “optionally” mean that the subsequently described event or circumstance may occur or may not occur, and that the description includes instances where the event or circumstance occurs as well as instances in which it does not.

As used herein, the term “prevents”, “preventing” and “prevention” refer to a method of preventing the onset of a disease and/or its attendant symptoms or barring a subject from acquiring a disease. As used herein, “prevent”, “preventing” and “prevention” also include delaying the onset of a disease and/or its attendant symptoms and reducing a subject's risk of acquiring a disease.

As used herein, the term “therapeutically effective amount” refers to that amount of the active ingredient being administered sufficient to prevent development of or alleviate to some extent one or more of the symptoms of the condition or disorder being treated.

As used herein, the term “treat”, “treating” and “treatment” refer to a method of alleviating or abrogating a disease and/or its attendant symptoms.

As used herein, the terms “composition” and “formulation” are interchangeable and have same meaning.

Each embodiment is provided by way of explanation of the invention and not by way of limitation of the invention. In fact, it will be apparent to those skilled in the art that various modifications and variations can be made to the compounds, and methods described herein without departing from the scope or spirit of the invention. For instance, features illustrated or described as part of one embodiment can be applied to another embodiment to yield a still further embodiment. Thus, it is intended that the present invention includes such modifications and variations and their equivalents. Other objects, features and aspects of the present invention are disclosed in or are obvious from, the following detailed description. It is to be understood by one of ordinary skill in the art that the present discussion is a description of exemplary embodiments only and is not to be construed as limiting the broader aspects of the present invention.

In an embodiment, the present disclosure provides a pharmaceutical composition (SACC) comprising at least one compound selected from the following formula:
(i)

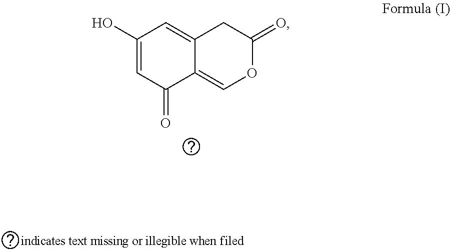 [SMILES: O=C1CC2=CC(O)=CC(=O)C2=CO1]

(ii)

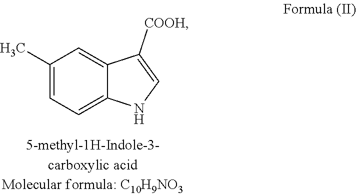

(iii)

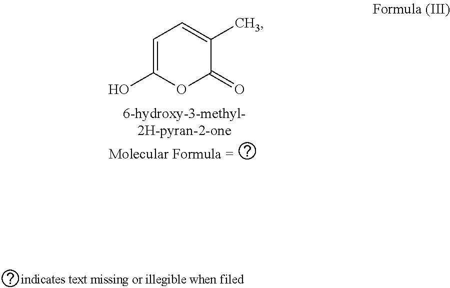

(iv)

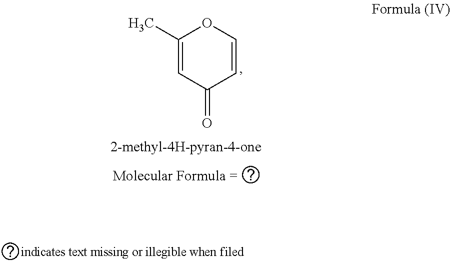

or a combination thereof.

In an embodiment, the present disclosure provides a pharmaceutical composition (SACC) comprising compounds of the following formula (I), formula (II), formula (III) and formula (IV):
(i)

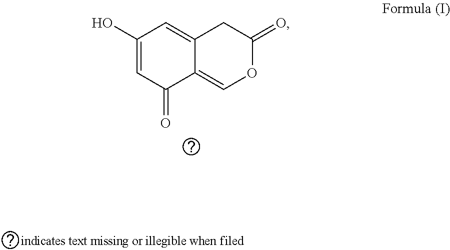 [SMILES: O=C1CC2=CC(O)=CC(=O)C2=CO1]

(ii)

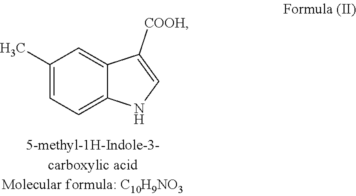

(iii)

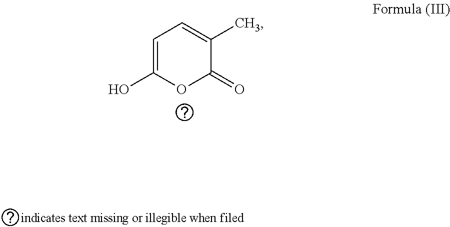

(iv)

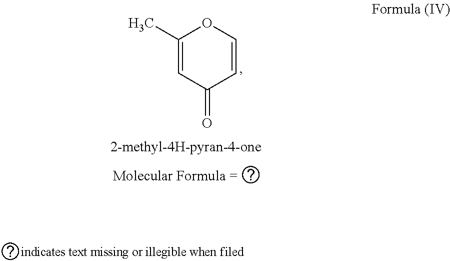

In an embodiment, the amount of compound of formula I in the pharmaceutical composition is in the range of 6 wt % to 18 wt %.

In another embodiment, the amount of compound of formula I the in pharmaceutical composition is 12.80 wt %.

In an embodiment, the amount of compound of formula II in the pharmaceutical composition is in the range of 8 wt % to 22 wt %.

In another embodiment, the amount of compound of formula II the pharmaceutical composition is 15.41 wt %.

In an embodiment, the amount of compound of formula III in the pharmaceutical composition is in the range of 2 wt % to 10 wt %.

In another embodiment, the amount of compound of formula III in the pharmaceutical composition is 6.48 wt %.

In an embodiment, the amount of compound of formula IV in the pharmaceutical composition is in the range of 1 wt % to 10 wt %.

In another embodiment, the amount of compound of formula IV in the pharmaceutical composition is 5.89 wt %.

In an embodiment, the present disclosure provides a compound represented by formula I:

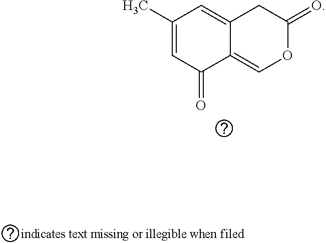

In an embodiment, the present disclosure provides a compound represented by formula II:

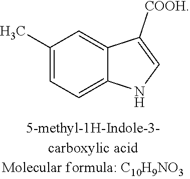

In an embodiment, the present disclosure provides a compound represented by formula III:

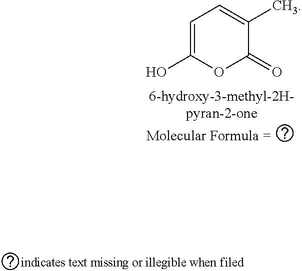

In an embodiment, the present disclosure provides a compound represented by formula IV:

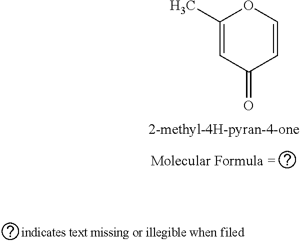

In an embodiment, the present disclosure provides a method of isolating the compounds of formula I, formula II, formula III, or formula IV, from barley extract:
(i)

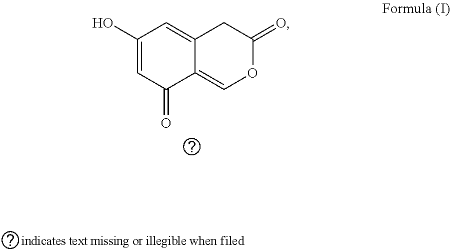 [SMILES: O=C1CC2=CC(O)=CC(=O)C2=CO1]

(ii)

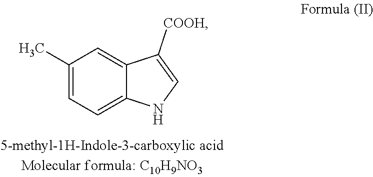

(iii)

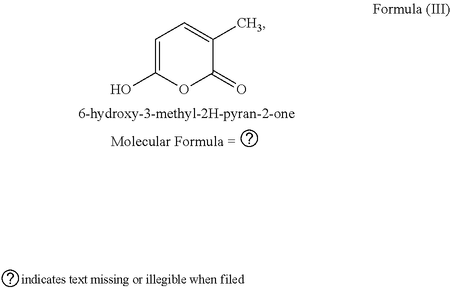

(iv)

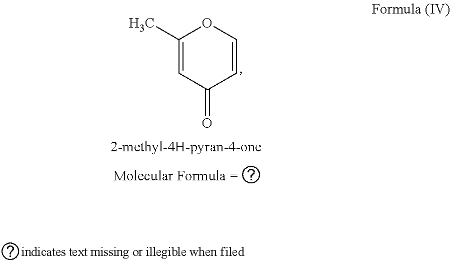

wherein, the method comprising the steps of:
performing the chromatography of barley extract by preparative HPLC method;
eluting the peak 2, peak 3, peak 4 and peak 5;
isolating the peaks obtained in step (ii) manually by repetitive injection to obtain fractions;
pooling, and concentrating the fractions at 35°; and
analysing the peaks and determining the structures of compounds of formula I, formula II, formula III, and formula IV.

In another embodiment, in step (i), preparative HPLC method comprises mobile phase A and mobile phase B.

In another embodiment, in step (ii), elution of peaks 2, 3, 4, and 5, is performed at about 14.3, 15.5, 16.4 and 23.0 minutes respectively.

In another embodiment, in step (iv), the fractions are pooled and concentrated at the temperature between the range of 25° C. to 45° C.

In another embodiment, in step (iv), the fractions are pooled and concentrated at the temperature of 35° C.

In another embodiment, in step (v), structure is determined by IR, Mass, NMR spectroscopy.

In an embodiment, the present disclosure provides HPLC method for the analysis of barley extract and the preparative HPLC method for the isolation of required peaks corresponding to the compounds of formulae (I), (II), (III) and (IV).

Band corresponding to the compounds have been eluted from the thin layer chromatography and confirmed that it is a single band. The HPLC analysis of the isolated compounds showed a single peak with a retention time (Rt) of 14.3, 15.5, 16.4 and 23.0 minutes. Also, the purity of the compound has been checked in HPLC which gave a single peak of purity between the range of 85% to 100%, preferably more than or equal to 87.9%, or more than or equal to 98.1% or more than or equal to 99.7%. Structures of the isolated compounds have been determined using LC-MS, ESI-MS, IR spectroscopy, and ¹H NMR.

In an embodiment, the present invention provides HPLC method for the analysis of barley extract and the preparative HPLC method for the isolation of required peaks corresponding to the compounds of formula I, formula II, formula III, or formula IV.

In an embodiment, the barley extract is obtained by the process comprising the steps of mixing barley grain flour with distilled water to get a mixture; stirring the mixture at an ambient temperature; and distilling the mixture to obtain the extract. In some instances of the extraction process, the mixture is kept at a temperature of about 27±3° C. for about 16±2 h. In certain embodiments, the mixture is distilled at a temperature of about 110±30° C. to obtain the extract. The extract thus obtained is kept at about 10±3° C. for about 1 h.

In an embodiment, the present disclosure provides a pharmaceutical composition comprising compound of formula I, formula II, formula III, or formula IV extracted from barley extract as an active ingredient, optionally admixed with a pharmaceutically acceptable carrier or excipient or diluent.

In an embodiment, the present disclosure provides an anti-cancer composition comprising at least one compound selected from formula (I), (II), (III) and (IV) or combination thereof and a pharmaceutically acceptable carrier/excipient.

In an embodiment, the present disclosure provides an anti-cancer composition comprising the compound of formula (I), (II), (III) and (IV) and a pharmaceutically acceptable carrier and/or excipient.

In an embodiment, the present disclosure provides a pharmaceutical formulation comprising a compound of formula (I), (II), (III) and (IV) or a pharmaceutically acceptable salt thereof, together with one or more pharmaceutically acceptable carriers and optionally one or more other therapeutic agents.

In another embodiment, the therapeutic agent is anticancer agent.

The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art.

In an embodiment, the present disclosure provides a method for formulating the disclosed compositions for pharmaceutical administration.

In one aspect of the embodiment, the administration includes intravenous, intrathecal, intramuscular, oral, and any other acceptable route of administration.

In an embodiment, the present disclosure provides a pharmaceutical composition comprising the compounds of formula (I), formula (II), formula (III), and formula (IV) obtained from Barley extract for inducing apoptosis and inhibiting the growth of pancreatic cancer cells irrespective of mutant p53.

In an embodiment, the present disclosure provides a pharmaceutical composition comprising the compounds of formula (I), formula (II), formula (III), and formula (IV) obtained from Barley extract for inhibiting the expression of HER-2.

In an embodiment, the present disclosure provides a pharmaceutical composition comprising the compounds of formula (I), formula (II), formula (III), and formula (IV) obtained from Barley extract for inducing the expression of pro-apoptotic protein Bax and inhibiting anti-apoptotic protein Bcl2.

In an embodiment, the present disclosure provides a pharmaceutical composition comprising the compounds of formula (I), formula (II), formula (III), and formula (IV) obtained from Barley extract for inducing the PARP protein cleavage.

In an embodiment, the present disclosure provides a pharmaceutical composition comprising the compounds of formula (I), formula (II), formula (III), and formula (IV) obtained from Barley extract for treatment of prostate cancer, triple negative breast cancer, invasive cancers, lung cancers, sarcoma, serous carcinoma, pancreatic cancer, and HER2 positive breast cancer, in a subject.

In an embodiment, the present disclosure provides a pharmaceutical composition for use in the manufacture of a medicament for inducing apoptosis and inhibiting the growth of pancreatic cancer cells irrespective of mutant p53.

In an embodiment, the present disclosure provides a pharmaceutical composition for use in the manufacture of a medicament for inhibiting the expression of HER-2.

In an embodiment, the present disclosure provides a pharmaceutical composition for use in the manufacture of a medicament for inducing the expression of pro-apoptotic protein Bax and inhibiting anti-apoptotic protein Bcl2.

In an embodiment, the present disclosure provides a pharmaceutical composition for use in the manufacture of a medicament for inducing the PARP protein cleavage.

In an embodiment, the present disclosure provides a pharmaceutical composition for use in the manufacture of a medicament for treatment of prostate cancer, triple negative breast cancer, invasive cancers, lung cancers, sarcoma, serous carcinoma, pancreatic cancer, and HER2 positive breast cancer, in a subject.

In an embodiment, the present disclosure provides a use of a pharmaceutical composition comprising a compound of formula (I), formula (II), formula (III), and formula (IV), for the manufacture of a medicament for treating cancer.

In an embodiment, the present disclosure provides a method of preventing or treating a cancer in a subject in need thereof comprising administering a therapeutically effective amount of a pharmaceutical composition comprising the compounds of formula (I), formula (II), formula (III), and formula (IV).

In an embodiment, the present disclosure provides a method of inducing apoptosis and inhibiting the growth of pancreatic cancer cells irrespective of mutant p53 in a subject in need thereof comprising administering a therapeutically effective amount of a pharmaceutical composition comprising a compound of formula (I), formula (II), formula (III), and formula (IV).

In an embodiment, the present disclosure provides a method of inhibiting the expression of HER-2 in a subject in need thereof comprising administering a therapeutically effective amount of a pharmaceutical composition comprising a compound of formula (I), formula (II), formula (III), and formula (IV).

In an embodiment, the present disclosure provides a method of inducing the expression of pro-apoptotic protein Bax and inhibiting anti-apoptotic protein Bcl2 in a subject in need thereof comprising administering a therapeutically effective amount of a pharmaceutical composition comprising a compound of formula (I), formula (II), formula (III), and formula (IV).

In an embodiment, the present disclosure provides a method of inducing the PARP protein cleavage in a subject in need thereof comprising administering a therapeutically effective amount of a pharmaceutical composition comprising a compound of formula (I), formula (II), formula (III), and formula (IV).

In an embodiment, the present disclosure provides a method of use of a pharmaceutical composition comprising a compound of formula (I), formula (II), formula (III), and formula (IV), wherein the method comprises inducing apoptosis and inhibiting the growth of pancreatic cancer cells.

In an embodiment, the present disclosure provides a method of use of a pharmaceutical composition comprising a compound of formula (I), formula (II), formula (III), and formula (IV), wherein the method comprises inhibiting the expression of HER-2.

In an embodiment, the present disclosure provides a method of use of a pharmaceutical composition comprising a compound of formula (I), formula (II), formula (III), and formula (IV), wherein the method comprises inducing the expression of pro-apoptotic protein Bax and inhibiting anti-apoptotic protein Bcl2.

In an embodiment, the present disclosure provides a method of use of a pharmaceutical composition comprising a compound of formula (I), formula (II), formula (III), and formula (IV), wherein the method comprises inducing the PARP protein cleavage.

In an embodiment, the present disclosure provides a method of use of a pharmaceutical composition comprising a compound of formula (I), formula (II), formula (III), and formula (IV), wherein the method comprises the treatment of prostate cancer, triple negative breast cancer, invasive cancers, lung cancers, sarcoma, serous carcinoma, pancreatic cancer, and HER2 positive breast cancer, in a subject.

In an embodiment, the present disclosure provides a compound of formula I, for use in the treatment of cancer.

In an embodiment, the present disclosure provides a compound of formula III, for use in the treatment of cancer.

In an embodiment, the present disclosure provides a use of a compound of formula I, for the manufacture of a medicament for treating cancer.

In an embodiment, the present disclosure provides a use of a compound of formula III, for the manufacture of a medicament for treating cancer.

In an embodiment, the present disclosure provides a pharmaceutical composition for use in treating a subject in need thereof.

In an embodiment, the present disclosure provides a pharmaceutical composition comprising a compound of formula (I), formula (II), formula (III), and formula (IV) for use as a medicament for treating cancer.

In an embodiment, the present disclosure provides a use of the pharmaceutical composition comprising a compound of formula (I), formula (II), formula (III), and formula (IV) in treating cancer.

In an embodiment, the present disclosure provides a pharmaceutical composition comprising a compound of formula (I), formula (II), formula (III), and formula (IV) for use as a medicament for inducing apoptosis, inducing the expression of pro-apoptotic protein Bax, and inducing the poly adenosine diphosphate-ribose polymerase (PARP) protein cleavage.

In an embodiment, the present disclosure provides a pharmaceutical composition comprising a compound of formula (I), formula (II), formula (III), and formula (IV) for use as a medicament for inhibiting the expression of human epidermal growth factor receptor-2 (HER-2), inhibiting anti-apoptotic protein Bcl2, inhibiting the growth of pancreatic cancer cells, and degrading the mutation of p53 and normalizing the wild type p53.

In an embodiment, the present disclosure provides a pharmaceutical composition comprising a compound of formula (I), formula (II), formula (III), and formula (IV) for use as a medicament for treatment of prostate cancer, triple negative breast cancer, invasive cancers, lung cancers, sarcoma, serous carcinoma, pancreatic cancer, and HER2 positive breast cancer, in a subject.

In one aspect of the present disclosure, the pharmaceutical composition comprising is an oral, parenteral, nasal, subcutaneous, intradermal, intramuscular, intravenous, intraarticular, and intramedullary, intraperitoneal, transmucosal, transdermal, rectal and topical composition.

In an embodiment, the present disclosure provides a method of inducing apoptosis, inducing the expression of pro-apoptotic protein Bax, and inducing the PARP protein cleavage in a subject comprising administering a therapeutically effective amount of a pharmaceutical composition comprising a compound of formula (I), formula (II), formula (III), and formula (IV).

In an embodiment, the present disclosure provides a method of inhibiting the expression of HER-2, inhibiting anti-apoptotic protein Bcl2, inhibiting the growth of pancreatic cancer cells, and degrading the mutation of p53 and normalizing the wild type p53, in a subject comprising administering a therapeutically effective amount of a pharmaceutical composition comprising a compound of formula (I), formula (II), formula (III), and formula (IV).

In another embodiment, the present disclosure provides a pharmaceutical composition wherein the subject is a human or a non-human mammal.

When administered to an animal, such as a human, the composition or the barley extract is preferably administered as a pharmaceutical composition comprising, for example, barley extract of the present disclosure or/and a pharmaceutically acceptable carrier. Pharmaceutically acceptable carriers are well known in the art and include, for example, aqueous solutions such as water or physiologically buffered saline or other solvents or vehicles such as glycols, glycerol, and oils such as olive oil or injectable organic esters.

In another embodiment, the present disclosure provides pharmaceutical compositions for human administration, particularly for invasive routes of administration (i.e., routes, such as injection or implantation, that circumvent transport or diffusion through an epithelial barrier), the aqueous solution is pyrogen-free or substantially pyrogen-free. The excipients can be chosen, for example, to effect delayed release of an agent or to selectively target one or more cells, tissues, or organs.

In another embodiment, the pharmaceutical composition can be in dosage unit form such as tablet, capsule (including sprinkle capsule and gelatin capsule), granule, and lyophile for reconstitution, powder, solution, syrup, suppository, injection, or the like. The composition can also be present in a transdermal delivery system, e.g., a skin patch. The composition can also be present in a solution suitable for topical administration, such as an eye drop.

The dosage of the active ingredient (barley extract) of the present disclosure varies depending on a patient's age, weight, or symptoms, as well as the compound's potency or therapeutic efficacy, the dosing regimen and/or treatment time. Generally, suitable routes of administration may, for example, include oral, eyedrop, rectal, transmucosal, topical or intestinal administration; parenteral delivery, including intramuscular, subcutaneous, intramedullary injections, as well as intrathecal, direct intraventricular, intravenous, intraperitoneal, intranasal, or intraocular injections.

The barley extract of the present disclosure may be administered in an amount of 0.2 mg, 0.5 mg or 1 mg up to 500 mg, 1 g, 2 g, 3 g, 4 g, 5 g, 6 g, 7 g, 8 g, 9 g, or 10 g, per dosage regimen. The dosage may be administered once per week, once per three days, once per two days, once per day, twice per day, three times per day or more often. In alternative embodiments, in certain adults the barley extract can be continuously administered by intravenous administration for a period of time designated by a physician. Since the dosage is affected by various conditions, an amount less than or greater than the dosage ranges contemplated about may be implemented in certain cases. A physician can readily determine the appropriate dosage for a patient undergoing therapeutic treatment.

The pharmaceutical compositions of the present disclosure may be manufactured in a manner that is itself known, e.g., by means of conventional mixing, dissolving, granulating, dragee-making, levigating, emulsifying, encapsulating, entrapping or compression processes. The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient.

The pharmaceutical compositions of the present disclosure may be used in the diagnostic imaging methods. The pharmaceutical composition of the present disclosure is preferably administered to the patient (e.g., a human) by intravenous injection. The formulations may conveniently be presented in unit dosage form and may be prepared by any of the methods well known in the art of pharmacy. All methods include the step of bringing into association a compound of the present invention or a pharmaceutically acceptable salt thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

The phrase “pharmaceutically acceptable salt” as used herein refers to prepared salts of pharmaceutically acceptable non-toxic acids, including inorganic acids and organic acids. Examples of physiologically or pharmaceutically acceptable salts of the compounds of the disclosure include salts derived from an appropriate base, such as an alkali metal (for example, sodium), an alkaline earth metal (for example, magnesium), ammonium and NX4+(wherein X is C1-C4 alkyl). Physiologically acceptable salts of a hydrogen atom or an amino group include salts of organic carboxylic acids such as acetic, benzoic, lactic, fumaric, tartaric, maleic, malonic, malic, isethionic, lactobionic and succinic acids; organic sulfonic acids, such as methanesulfonic, e thanesulfonic, benzenesulfonic and p-toluenesulfonic acids; and inorganic acids, such as hydrochloric, sulfuric, phosphoric and sulfamic acids. Physiologically acceptable salts of a compound of a hydroxy group include the anion of said compound in combination with a suitable cation such as Na+ and NX/(wherein X is independently selected from H or a C1-C4 alkyl group).

For therapeutic use, salts of active ingredients of the compounds of the disclosure will typically be physiologically acceptable, i.e. they will be salts derived from a physiologically acceptable acid or base. However, salts of acids or bases which are not physiologically acceptable may also find use, for example, in the preparation or purification of a physiologically acceptable compound. All salts, whether or not derived form a physiologically acceptable acid or base, are within the scope of the present disclosure.

Also included within the scope of this disclosure are the salts of the parental compounds with one or more amino acids. Any of the natural or unnatural amino acids are suitable, especially the naturally occurring amino acids found as protein components, although the amino acid typically is one bearing a side chain with a basic or acidic group, e.g., lysine, arginine or glutamic acid, or a neutral group such as glycine, serine, threonine, alanine, isoleucine, or leucine.

The phrase “pharmaceutically acceptable carrier” as used herein means a pharmaceutically acceptable material, composition, or vehicle, such as a liquid or solid filler, diluent, excipient, solvent, or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin or as an eye drop). The barley extract may also be formulated for inhalation. In yet another embodiment, barley extract may be simply dissolved or suspended in sterile water or double distilled water (DDW).

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Formulations of the present disclosure suitable for oral administration may be in the form of capsules (including sprinkle capsules and gelatin capsules), cachets, pills, tablets, lozenges (using a flavored basis, usually sucrose and acacia or tragacanth), lyophile, powders, granules or as a solution or a suspension in an aqueous or non-aqueous liquid or as an oil-in-water or water-in-oil liquid emulsion or as an elixir or syrup or as pastilles (using an inert base, such as gelatin and glycerin or sucrose and acacia) and/or as mouth washes and the like, each containing a predetermined amount of barley extract of 5 the present disclosure as an active ingredient.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate and/or any of the following: (1) fillers or extenders, such as starches, lactose, sucrose, glucose, mannitol and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate and mixtures thereof; (10) complexing agents, such as, modified and unmodified cyclodextrins; and (11) coloring agents.

In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms useful for oral administration include pharmaceutically acceptable emulsions, lyophiles for reconstitution, microemulsions, suspensions, solutions, syrups, and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active ingredient, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth and mixtures thereof.

Formulations of the pharmaceutical compositions for rectal, vaginal or urethral administration may be presented as a suppository, which may be prepared by mixing one or more active compounds with one or more suitable non irritating excipients or carriers comprising, for example, cocoa butter, polyethylene glycol, a suppository wax or a salicylate and which is solid at room temperature, but liquid at body temperature and, therefore, will melt in the rectum or vaginal cavity and release the active compound.

Formulations of the pharmaceutical compositions for administration to the mouth may be presented as a mouthwash or an oral spray or an oral ointment.

Alternatively, or additionally, compositions can be formulated for delivery via a catheter, stent, wire, or other intraluminal device. Delivery via such devices may be especially useful for delivery to the bladder, urethra, ureter, rectum, or intestine.

The pharmaceutical composition of the present disclosure may be used alone (monotherapy) or conjointly with one or more other methods/compositions (combined therapy).

### ABBREVIATIONS

The following abbreviations are used in the present disclosure:
° C. degree Celsius
HPLC High performance liquid chromatography
h hour(s)
mL millilitre
THF Tetrahydrofuran
Rt, Retention time
TLC Thin layer chromatography
NMR Nuclear Magnetic Resonance
MHz Mega Hertz
s singlet
d doublet
t triplet
m multiplet
H Proton
MS Mass Spectrometry
LC-MS Liquid Chromatography Mass Spectrometry
IV or i.v. Intravenous administration
HEPES 4-(2-hydroxyethyl)-1-piperazineethanesulfonic acid)
ATP Adenosine Tri Phosphate
DTT Dithiothreitol
EGTA Eethylene glycol bis(2-aminoethyl) ether-N,N,N,N-tetraacetic acid)
DAPK1 Death Associated Protein Kinase
PKD: Protein Kinase D
CAMKK2: Calcium/calmodulin-dependent protein kinase kinase 2
PKCδ Protein Kinase C-delta
FTIR Fourier Transform infrared spectroscopy
ppm parts per million
PARP Poly adenosine diphosphate-ribose polymerase

The present disclosure now being generally described, it will be more readily understood by reference to the following examples, which are included merely for purposes of illustration of certain aspects and embodiments of the present disclosure and are not intended to limit the present disclosure in any way.

### EXAMPLES

### Instrument and Material

The following instruments are used in the present invention:

| | |
|---|---|
| Preparative HPLC: | Agilent semi preparative HPLC, |
| NMR: | 400 MHz NMR Varian Make (Agilent) |
| FTIR: | Perkin Elmer 100 series |
| HPLC: | Waters HPLC with Empower software |
| LC-MS: | Agilent Single Quadrupole with ESI |
| Balance: | Sartorius |
| Column: | Inertsil C18 (250 × 4.6 mm, 5 μm) for HPLC analysis and |
| | Inertsil C18 (250 × 19 mm, 5 μm) for preparative HPLC |

The following materials are used in the present invention:

| | |
|---|---|
| Acetonitrile: | HPLC Grade |
| Methanol: | HPLC Grade |
| Trifluoro acetic acid: | A. R. Grade |
| Dichloromethane (DCM): | HPLC Grade |
| Water: | Mili-Q water |

### Preparation of Barley Extract

One kilogram of barley flour, derived from ground barley grains which are obtained from India, is placed in a glass vessel equipped with a standard joint. To this, 2 litres of double-distilled water are added, and the mixture is thoroughly stirred and vigorously shaken. The glass vessel is then securely covered and placed in an incubator at 27±3° C. for 16 h. After maturation, the vessel cover is removed, and the vessel is positioned on a heating system (rotamantle with a heating element and thermostat, insulated with glass wool to prevent direct heat contact with glass flasks). The vessel is equipped with a distillation set featuring a proper cooling system, with the optimal distillation temperature maintained at 110±30° C.

The liquid obtained during the distillation process is passed over a cotton bed fitted in funnels and collected in flasks, typically in 3-4 fractions, each approximately 400 ml. These fractions are securely covered and stored at a temperature of 10±3° C. for 1 h. Subsequently, the flasks are returned to normal room temperature, which may vary depending on the season. The resulting solution constitutes the pharmaceutical preparation intended for use.

### Example 2: Isolation of Compounds from Barley Extract

The compounds are isolated from the barley extract using HPLC techniques. Purity of the compounds have been determined.

### 1. Method Details for Isolation of Fractions:

1.1 HPLC Method Details for the Analysis of Barley Extract (B. No. KWO 037)

| | |
|---|---|
| Mobile phase A: | 0.1% TFA in water |
| Mobile phase B: | 0.1% TFA in Methanol:ACN (80:20 v/v) |

Following chromatographic conditions are followed for analysis of barley extract:

| | |
|---|---|
| Column: | Inertsil ODS 3V (250 mm × 4.6 mm, 5 μm) |
| Injection volume: | 10 μL |
| Flow rate: | 1.0 mL/minute |
| Column temperature: | 25° C. |
| Detection wavelength: | Max plot, 280, 254 and 230 nm |
| Run time: | 45 minutes |
| Test concentration: | As such |
| Elution mode: | Gradient mode/program is shown in Table 1 |

TABLE 1
Gradient

| Time (Min.) | (% Mobile phase A) | (% Mobile phase B) |
|---|---|---|
| 0 | 10 | 90 |
| 2 | 10 | 90 |
| 15 | 20 | 80 |
| 25 | 80 | 20 |
| 35 | 80 | 20 |
| 38 | 10 | 90 |
| 45 | 10 | 90 |

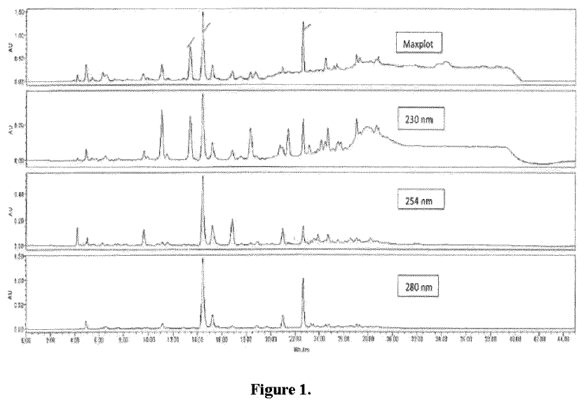

Typical HPLC chromatograms at different wavelengths of UV (in nm) is shown in FIG. 1.

### 1.2 Preparative HPLC Method Details for the Isolation of Peaks

The following HPLC method is used for isolation of peaks:

| | |
|---|---|
| Mobile phase A: | 0.1% TFA in water |
| Mobile phase B: | 0.1% TFA in Methanol:ACN (80:20 v/v) |

Following chromatographic conditions are followed for isolation of peaks:

| | |
|---|---|
| Column: | Inertsil C18 (250 mm × 19 mm, 5 μm) |
| Flow rate: | 15.0 mL/minute |
| Column temperature: | 25° C. |
| Detection wavelength: | 230 nm |
| Run time: | 45 minutes |
| Test concentration: | As such |

### Procedure:

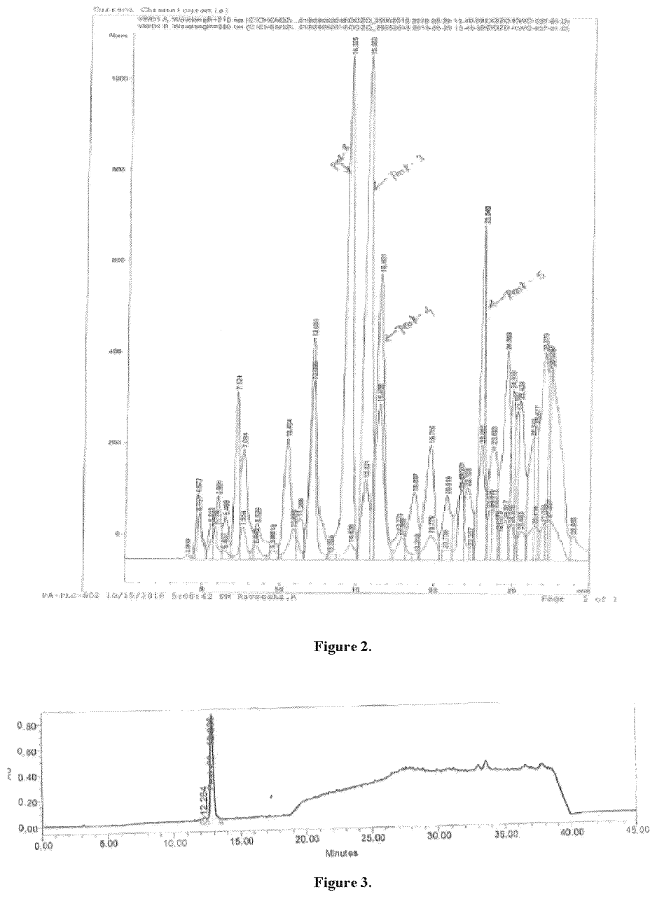

When the barley extract is chromatographed with the above method, peak 2, peak 3, peak 4 and peak 5 are eluted at about 14.3, 15.5, 16.4 and 23.0 minutes respectively as shown in FIG. 2. The peaks are isolated manually by repetitive injection and the fractions are pooled and concentrated at 35°.

### a) Isolation and Analysis of Isolated Peak 2

The peak 2 is isolated manually by repetitive injection and the fractions are pooled and concentrated at 35° as per the chromatographic condition mentioned in section 1.2.

### Details of HPLC Method Details:

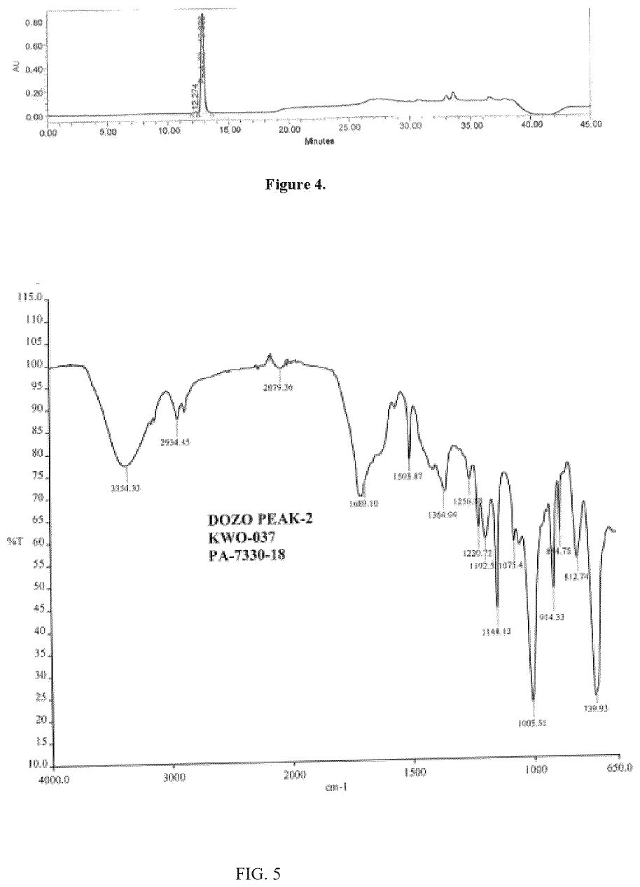

Chromatographic conditions are provided under section 1.1. The HPLC chromatogram (FIGS. 3-4) indicates 98.17% area purity at max plot. The peak purity angle and peak purity threshold is 5.893 and 6.041, respectively, indicating the compound peak is highly pure and the purity measured by HPLC is reliable.

The structure of the compound corresponding to peak2 has been assigned by the following studies: Infrared spectroscopy (IR), Mass spectrometry (MS) and 1H-NMR.

All analyses have been performed using the isolated peak 2 in dried form, isolated by preparative HPLC and concentrated.

### Infrared Spectroscopy (IR):

IR spectrum is generated by dispersing with KBr and recorded. FIG. 5 shows a representative IR spectrum for the peak 2. IR spectra for peak-2 is mentioned in Table 2 below:

TABLE 2
IR spectra for peak 2:

| SI. | Frequency | Assignment |
|---|---|---|
| No. | (cm⁻¹) | (Functional group) |
| 1 | 3354.33 | O—H Stretching |
| 2 | 2934.45 | C—H Stretching |
| 3 | 1689.10 | C═O Stretching |
| 4 | 1364.04 | C—H bending |
| 5 | 1220.72 | C—O Stretching |

### Mass Spectrometry (MS):

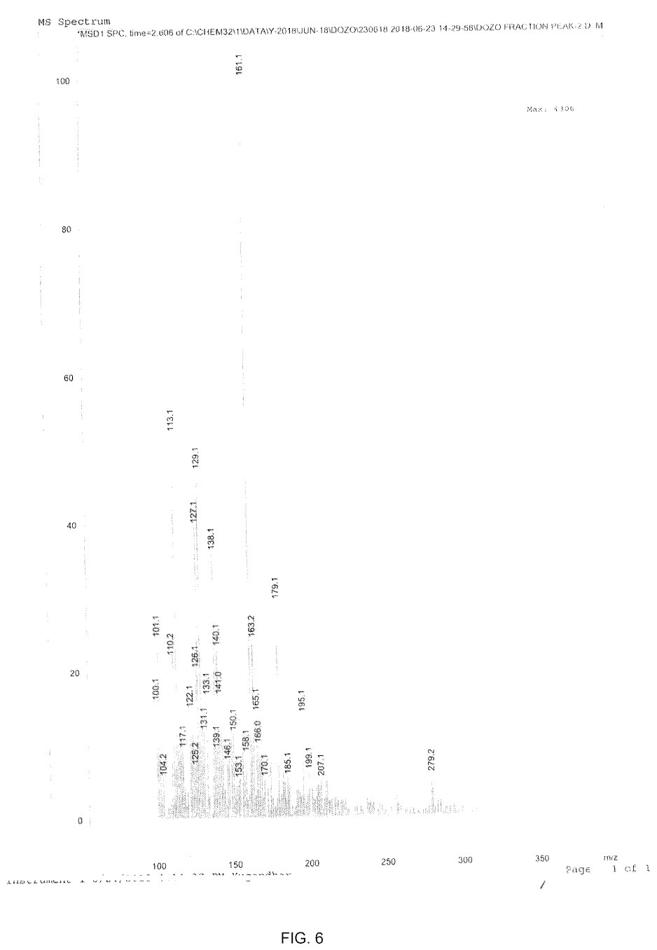

This spectrometry is performed using electrospray ionisation (ESI)/APCI and quadruple analyser. The spectrum is acquired in positive ion mode. FIG. 6 represents a representative MS spectrum. The MS peak of 179.1 mlz [M+H] spectra correspond to the molecular weight of 178 amu.

### NMR Spectroscopy

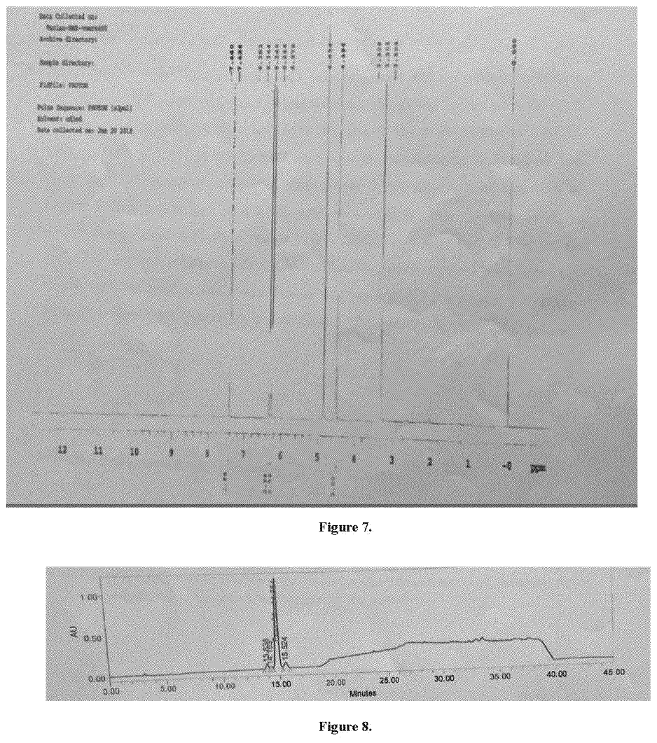

This spectrometry is performed in deuterated methanol (CD₃OD) as solvent. FIG. 7 represents a ¹H NMR spectrum of peak 2. ¹H-NMR (400 MHz, CD₃OD, ppm) spectra for peak-2 is mentioned in Table 3 below:

TABLE 3
NMR spectra for peak 2:

| Proton shift (δ) | Multiplicity | Number of protons |
|---|---|---|
| 4.496 | s | 2H |
| 6.27 to 6.286 | d | 1H |
| 6.340 to 6.352 | m | 1H |
| 7.438 t07.440 | Broad singlet | 1H |

### Structure Determination of Peak 2:

Based on result obtained by LC-MS, IR and NMR, the basic structure of the compound corresponds to peak 2 is identified as formula (I). The LC-MS analysis data of the compound exhibited the molecular ion peak (M+1) at 179.1 m/z corresponding to the mass of 178 amu (FIG. 6). The IR absorption at 3354.33, 2934.45, 1689.10, 1364.04 and 1220.72 cm-1 revealed the compound contained OH, CH, C═O, CH bending and c-o groups, respectively. In the proton NMR spectrum of peak-2, a singlet appeared at δ 4.496 ppm accounted for two protons, which is corresponding to the methylene groups (—CH2). Signals at δ 6.27 to 6.286, 6.340 to 6.352 and 7.438 to 7.440 ppm confirm the aromatic CH protons. On the basis of the LC-MS, IR and NMR spectral data it is inferred that the that the peak-2 is 6-hydroxy-3H-isochromene-3, 8(4H)-dione and the structure of the peak 2 is as below:

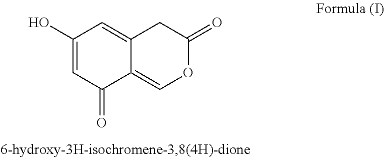 [SMILES: O=C1CC2=CC(O)=CC(=O)C2=CO1]

### b) Isolation and Analysis of Isolated Peak 3

The peak 3 is isolated manually by repetitive injection and the fractions are pooled and concentrated at 350 as per the chromatographic condition mentioned in section 1.2.

### Structure Determination of Peak 3:

Based on result obtained by LC-MS, IR and NMR, the basic structure of the compound corresponds to peak 3 is identified as formula (II). The LC-MS analysis data of the compound exhibited the molecular ion peak (M+1) corresponding to the mass of 191 amu. On the basis of the LC-MS, IR and NMR spectral data it is inferred that the that the peak-3 is 5-methyl-1H-indole-3-carboxylic acid and the structure of the peak 3 is as below:

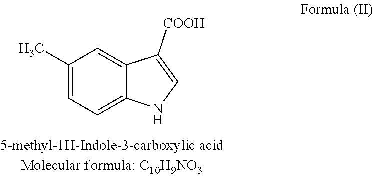

### c) Isolation and Analysis of Isolated Peak 4

The peak 4 is isolated manually by repetitive injection and the fractions are pooled and concentrated at 35° as per the chromatographic condition mentioned in section 1.2.

### Details of HPLC Method Details:

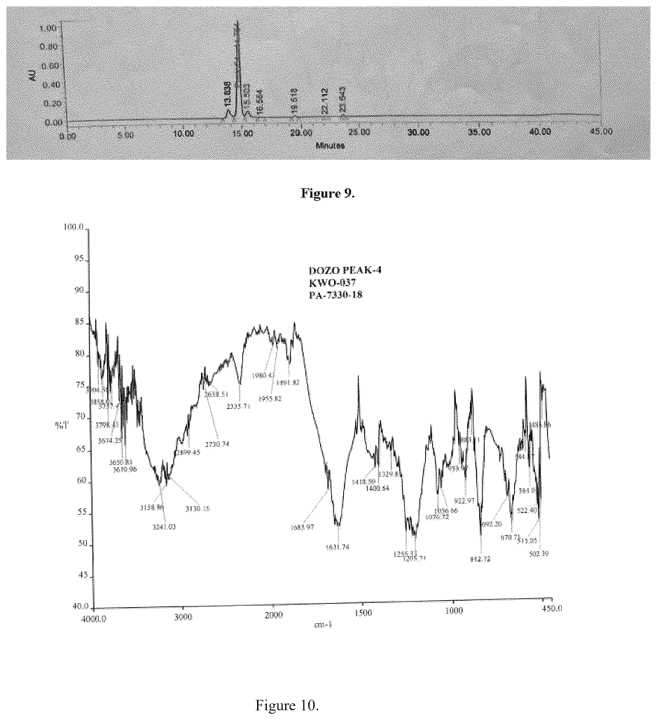

The HPLC chromatogram (FIGS. 8-9) indicates 87.98% area purity at max plot. The peak purity angle and peak purity threshold is 0.710 and 2.010, respectively, indicating the compound peak is highly pure and the purity measured by HPLC is reliable.

The structure of the compound corresponding to peak-4 has been assigned by the following studies: Infrared spectroscopy (IR), Mass spectrometry (MS) and 1H-NMR. All analyses have been performed using the isolated peak-4 in dried form, isolated by preparative HPLC and concentrated.

### Infrared Spectroscopy (IR):

IR spectrum is generated by dispersing with KBr and recorded. FIG. 10 shows a representative IR spectrum for the peak 4. IR spectra for peak-4 is mentioned below:

TABLE 4
IR assignment for peak 4

| SI. | Frequency | Assignment |
|---|---|---|
| No. | (cm⁻¹) | (Functional group) |
| 1 | 3241.03 | O—H Stretching |
| 2 | 3158.86 | C—H Stretching |
| 3 | 1631.74 | C═O Stretching |
| 4 | 1255.33 | C—O Stretching |

### Mass Spectrometry

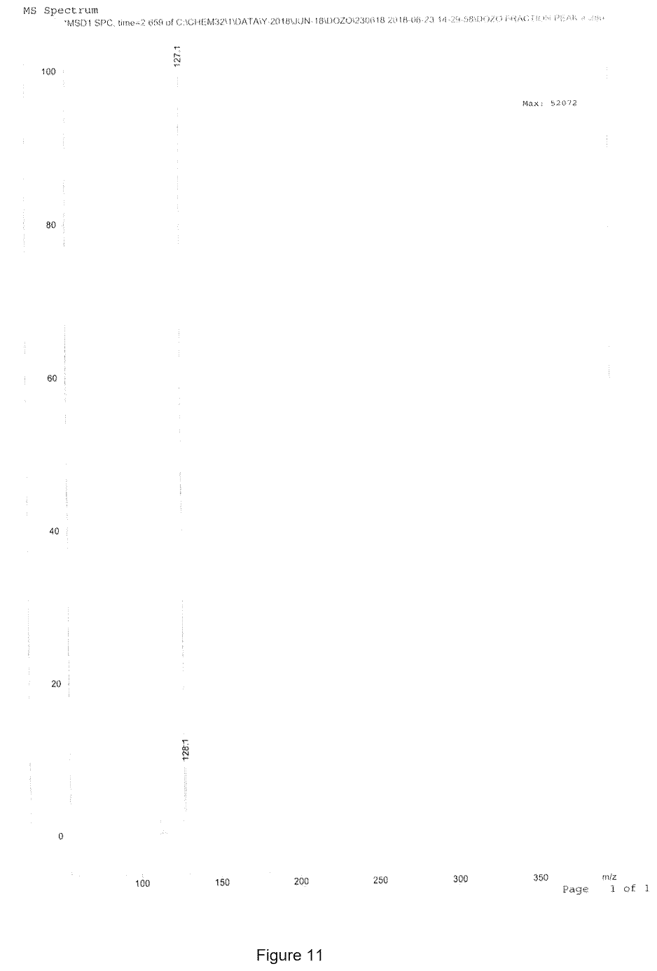

This spectrometry is performed using electrospray ionisation (ESI)/APCI and quadruple analyser. The spectrum is acquired in positive ion mode. FIG. 11 represents a representative MS spectrum. The MS peak of 127.1 m/z [M+H] spectra correspond to the molecular weight of 126 amu confirmed in the spectrum.

### NMR Spectroscopy

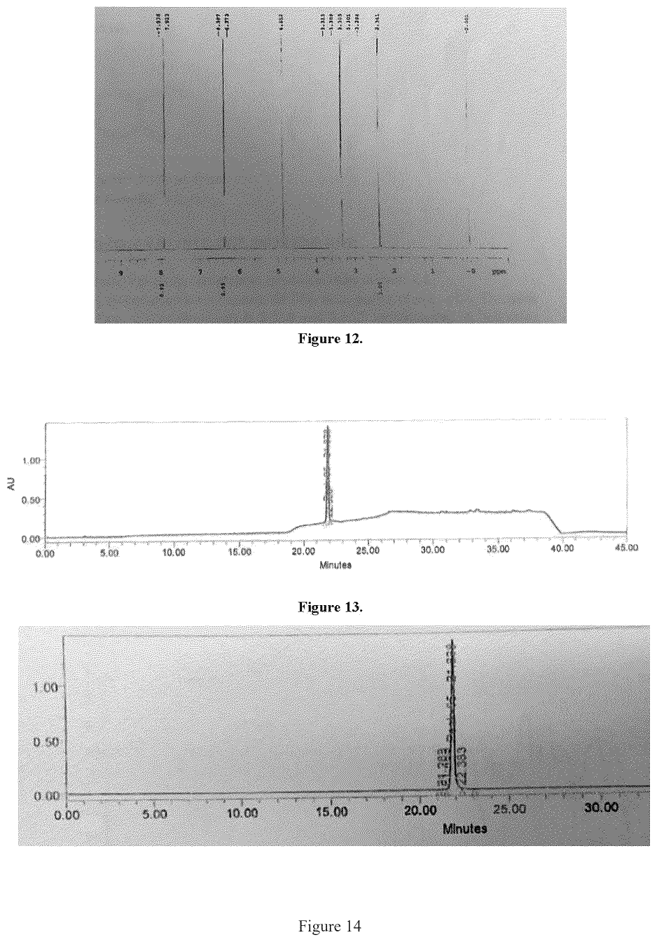

This spectroscopy is performed in deuterated methanol (CD₃OD) as solvent. FIG. 12 represents a representative NMR spectrum. ¹H-NMR (400 MHz, CD₃OD, ppm)

TABLE 5
NMR spectra for peak 4:

| Proton shift (δ) | Multiplicity | Number of protons |
|---|---|---|
| 2.341 | s | 3H |
| 6.373 to 6.387 | d | 1H |
| 7.922 to 7.936 | d | 1H |

### Structure Determination of Peak 4:

Based on result obtained by LC-MS, IR and NMR, the basic structure of the compound corresponds to peak 4 is identified as formula (III). The LC-MS analysis data of the compound exhibited the molecular ion peak (M+1) at 127.1 m/z corresponding to the mass of 126 amu (FIG. 12). The IR absorption at 3241.03, 3158.86, 1631.74 and 1255.33 cm⁻¹ revealed the compound contained OH, CH, C═O and C—O groups, respectively. In the proton NMR spectrum of peak-4, a singlet appeared at δ 2.341 ppm accounted for three protons, which is corresponding to the methyl group (—CH3). Signals at δ 6.373 to 6.387 and 7.922 to 7.936 ppm confirms the aromatic CH protons. Based on the LC-MS/MS, IR and NMR spectral data it is inferred that the that the peak-4 is 6-hydroxy-3-methyl-2H-pyran-2-one and the structure of the peak-4 is as below:

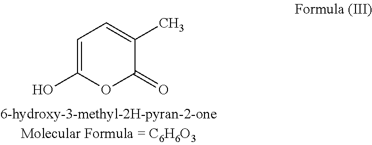

### d) Isolation and Analysis of Isolated Peak 5

The peak 5 is isolated manually by repetitive injection and the fractions are pooled and concentrated at 350 as per the chromatographic condition mentioned in section 1.2.

### Details of HPLC Method:

The HPLC chromatogram (FIGS. 13-14) indicates 99.74% area purity at max plot. The peak purity angle and peak purity threshold is 2.244 and 2.512, respectively, is indicating the compound peak is highly pure and the purity measured by HPLC is reliable.

The structure of the peak-5 has been assigned by the following studies: Infrared spectroscopy (IR), Mass spectrometry (MS) and 1H-NMR. All analyses have been performed using the isolated peak-5 in dried form, isolated by preparative HPLC and concentrated.

### Infrared Spectroscopy (IR):

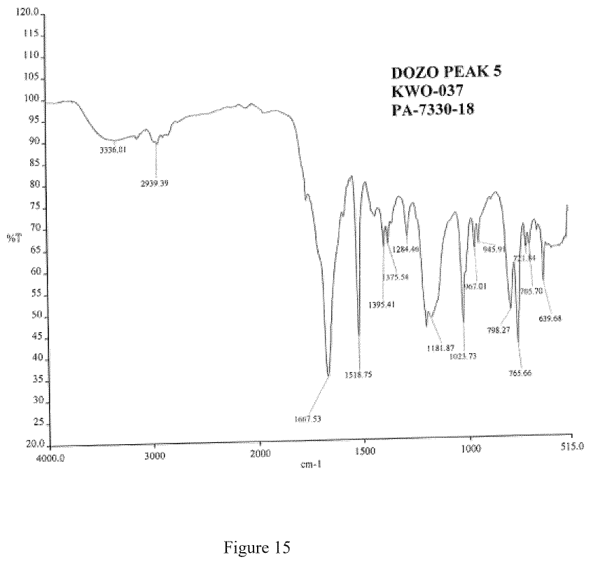

IR spectrum is generated by dispersing with KBr and recorded. FIG. 15 shows a representative IR spectrum for the peak 5. IR spectra for peak-5 is mentioned below:

TABLE 6
IR assignment for peak 5

| SI. | Frequency | Assignment |
|---|---|---|
| No. | (cm⁻¹) | (Functional group) |
| 1 | 2939.39 | C—H Stretching |
| 2 | 1667.53 | C═O Stretching |
| 3 | 1518.75 | C═C Stretching |
| 4 | 1395.41 | C—O bending |
| 5 | 1181.87 | C—O Stretching |

### Mass Spectrometry

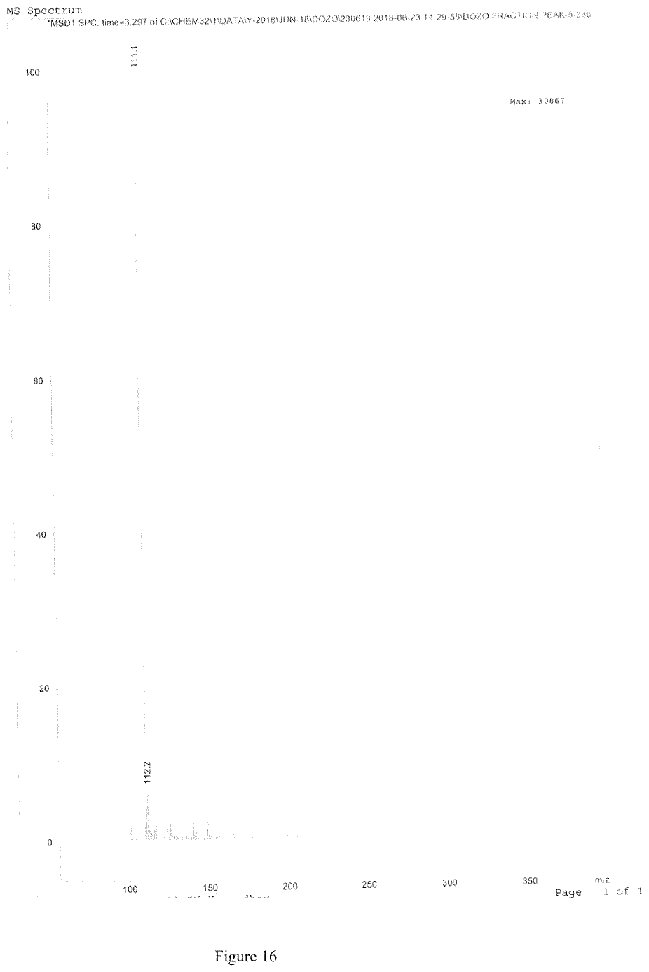

This spectrometry is performed using electrospray ionisation (ESI)/APCI and quadruple analyser. The spectrum is acquired in positive ion mode. The FIG. 16 represents a representative MS spectrum. The MS peak of 111.1 m/z [M+H] spectra correspond to the molecular weight of 110 amu confirmed in the spectrum.

### NMR Spectroscopy

This spectroscopy is performed in deuterated methanol (CD₃OD) as solvent. FIG. 17 represents a representative NMR spectrum. ¹H-NMR (400 MHz, CD₃OD, ppm) spectra for peak 5 is mentioned in Table 7 below:

TABLE 7
NMR spectra for peak 5:

| Proton shift (δ) | Multiplicity | Number of protons |
|---|---|---|
| 2.256 | s | 3H |
| 5.326 | s | 1H |
| 5.968-5.963 | d | 1H |
| 6.270-6.262 | d | 1H |

### Structure Determination of Peak 5:

Based on result obtained by LC-MS, IR and NMR, the basic structure of the compound corresponds to peak 5 is identified as formula (IV). The LC-MS analysis data of the compound exhibited the molecular ion peak (M+1) at 111.1 m/z corresponding to the mass of 111 amu (FIG. 16). The IR absorption at 2939.39, 1667.53, 1518.75, 1395.41 and 1181.87 cm⁻¹ revealed the compound contained CH, C═O, C═C stretching, C—H bending and C—O groups, respectively. In the proton NMR spectrum of peak-5, a singlet appeared at δ 2.256 ppm accounted for three protons, which is corresponding to the methyl group (—CH3). Signals at δ 5.326, 5.968 to 5.963 and 6.270 to 6.262 ppm confirm the aromatic CH protons. Based on the LC-MS, IR and NMR spectral data it is inferred that the that the peak-5 is 2-methyl-4H-pyran-4-one and the structure of the peak-5 is assigned as below:

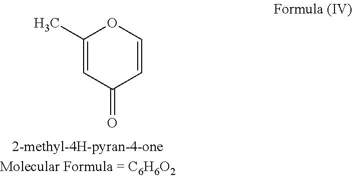

### Biological Activity of the Composition

### Example 3: Preparation of Pharmaceutical Composition

The pharmaceutical composition comprising the compounds of formula (I), formula (II), formula (III), and formula (IV) obtained from barley extract is prepared by obtaining a high-quality barley extract through a suitable extraction and purification process. The obtained barley extract is kept as liquid or dried form for intended use.

### Example 4: Experiment for Kinase Reactions

### Reaction Set Up:

2.5 μL barley extract (SACC Extract) ( 1/30 final dilution, 1/120 final dilution, or titration);
5.0 μL Enzyme dilution buffer (EDB) (1×) or Kinase (5× in EDB);
10-minute preincubation at room temperature;
17.5 μL Reaction Mix with ATP & C(Sx) Substrate (Previously preincubated for 5 min at 30° C.);
25 μL Final reaction volume;

Reaction was run at 30° C. for 120 or 240 minutes.

### Reaction Conditions:

54 mM HEPES, pH 7.5
1 mM ATP
1 mM DTT (no DTT for ASK1)
0.012% Brij-35
1% glycerol
0.52 mM EGTA (no EGTA for CAMKK2, DAPK1, DAPK2, PKC6, PKD1, PKD2, PKD3) 0.4 mM CaCl₂), 5 ng/μl Calmodulin (CAMKK2, DAPK1, DAPK2, PKD1, PKD2, PKD3 only)
3.8 μM Diacylglycerol, 140 μM Phosphatidylserine (PKCδ only)
250 μM MnCl₂ (HER2 only)
10 mM MgCl₂
15 μM C(Sx) Substrate
0.5-20 nM Kinase

### Notes:

Enzyme Dilution Buffer (EDB): 20 mM HEPES, pH 7.5, 0.01% Brij-35, 0.1 mM EGTA (no EGTA for CAMKK2, DAPK1, DAPK2, PKC6, PKD1, PKD2, PKD3), 5% Glycerol, 1 mg/mL Bovine Serum Albumin, 1 mM DTT (no DTT for ASK1).
Reactions were run in Corning, 384-well, white flat round bottom polystyrene NBS microplates (Cat. #3824) after sealing using optically clear adhesive film [TopSealA-Plus plate seal (PerkinElmer, Cat #6050185).

### Kinases Tested

| | Kinase | Enzyme Source (Cat. #) | Sensor |
|---|---|---|---|
| 1 | 8 nM AAK1 | Thermo (A33370) | AQT0759 |
| 2 | 2 nM ABL1 | Carna (08-001) | AQT0032 |
| 3 | 2 nM ABL2 (ARG) | Carna (08-102) | AQT0032 |
| 4 | 2 nM ADRBK1 (GRK2) | Thermo (PV3361) | AQT0641 |
| 5 | 20 nM CAMKK2 | Carna (05-108) | AQT0699 |
| 6 | 10 nM CDK1/CycE1 | Proqinase (0134-0055-1) | AQT0297 |
| 7 | 2 nM CDK2/CycA2 | Carna (04-103) | AQT0297 |
| 8 | 2 nM CDK2/CycE1 | Carna (04-165) | AQT0297 |
| 9 | 10 nM CSNK2A1 (CK2α1) | Thermo (PV3248) | AQT0696 |
| 10 | 2 nM DAPK1 | SignalChem (D01-11G) | AQT0240 |
| 11 | 5 nM DAPK2 (DRP-1) | SignalChem (D02-10G) | AQT0240 |
| 12 | 20 nM DDR1/2 nM SRC | Carna (08-113)/(08-173) | AQT0259 |
| 13 | 10 nM DDR2/1 nM SRC | Carna (08-114)/(08-173) | AQT0259 |
| 14 | 2.5 nM DYRK1A | Carna (04-130) | AQT0260 |
| 15 | 2.5 nM FER | Carna (08-139) | AQT0101 |
| 16 | 2.5 nM FLT4 | Carna (08-190) | AQT0101 |
| 17 | 20 nM GAK | Carna (05-121) | AQT0766 |
| 18 | 8 nM ERBB2 (HER2) | BPS (BS40230) | AQT0001 |
| 19 | 5 nM HIPK2 | Carna (04-136) | AQT0260 |
| 20 | 2.5 nM IGF1R | Carna (08-141) | AQT0101 |
| 21 | 2 nM IRAK1 | Carna (09-101) | AQT0326 |
| 22 | 2 nM IRAK4 | Carna (09-145) | AQT0326 |
| 23 | 20 nM LIMK1 | Carna (09-105) | AQT0459 |
| 24 | 20 nM MAP3K5 (ASK1) | Thermo (PV3809) | AQT0557 |
| 25 | 20 nM MAP3K12 (biotin-DLK) | Carna (09-411-20N) | AQT0645 |
| 26 | 2 nM MAP3K8 (Tpl2, COT) | Carna (07-301) | AQT0742 |
| 27 | 4 nM MAP3K9 (MLK1) | Carna (09-115) | AQT0645 |
| 28 | 2.5 nM MAP4K5 (KHS1) | SignalChem (M27-10G) | AQT0178 |
| 29 | 3 nM MAPK (JNK1) | Carna (04-163) | AQT0365 |
| 30 | 3 nM MAPK9 (JNK2) | Carna (04-164) | AQT0365 |
| 31 | 3 nM MAPK10 (JNK3) | Carna (04-150) | AQT0365 |
| 32 | 1 nM MAPK11 (p38β) | Carna (04-153) | AQT0376 |
| 33 | 1 nM MAPK12 (p38γ) | Carna (04-155) | AQT0376 |
| 34 | 1 nM MAPK13 (p38δ) | Carna (04-154) | AQT0376 |
| 35 | 1 nM MAPK14 (p38α) | Carna (04-152) | AQT0376 |
| 36 | 3 nM MAPK7 (ERK5) | Carna (04-146) | AQT0373 |
| 37 | 2.5 nM MAPKAPK2 (MK2) | Carna (02-142) | AQT0425 |
| 38 | 1 nM MERTK (MER) | Carna (08-108) | AQT0101 |
| 39 | 20 nM PAK1 | Carna (07-123) | AQT0269 |
| 40 | 20 nM PAK2 | Carna (07-124) | AQT0269 |
| 41 | 1 nM PRKCD (PKCδ [delta]) | Carna (01-135) | AQT0462 |
| 42 | 20 nM PRKD1 (PKD1) | Carna (02-157) | AQT0232 |
| 43 | 20 nM PRKD2 (PKD2) | Carna (02-158) | AQT0232 |
| 44 | 20 nM PRKD3 (PKD3) | Carna (02-159) | AQT0232 |
| 45 | 15 nM RIPK2 | Thermo (PV4213) | AQT0209 |
| 46 | 3 nM ROCK1 | Carna (01-109) | AQT0231 |
| 47 | 3 nM ROCK2 | Carna (01-110) | AQT0231 |
| 48 | 5 nM SRPK2 | Carna (04-161) | AQT0360 |
| 49 | 3 nM TNIK (MAP4K family) | Carna (07-138) | AQT0181 |

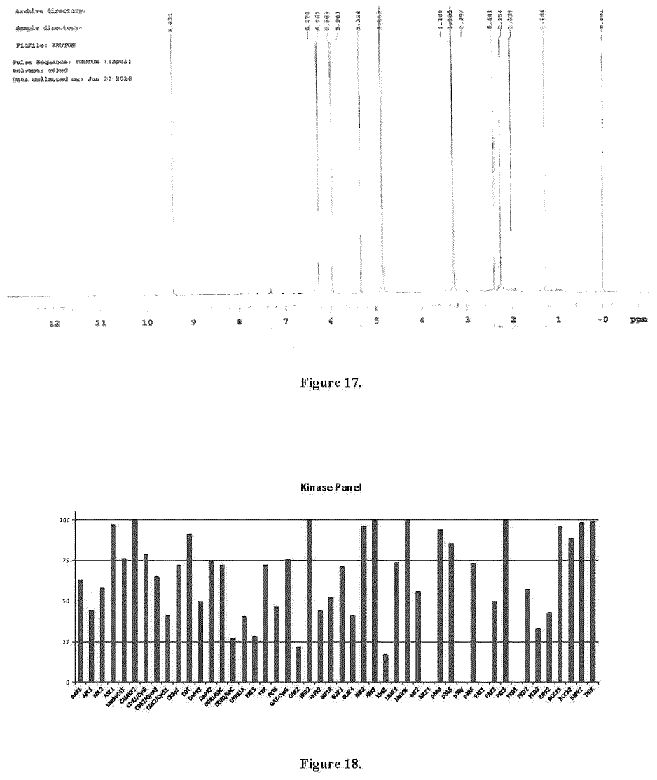

In FIG. 18, the Histogram (for 1/30 Final Dilution SACC) depicts the inhibitory effects of a 1:30 dilution of the crude compound on various kinases. The bars in the histogram represent the level of inhibition, with larger bars indicating greater inhibition. Notably, at this dilution, four kinases exhibit no inhibition, two kinases show less than 25% inhibition, while a substantial number of 30 kinases (61%) demonstrate significant inhibition, exceeding 50%.

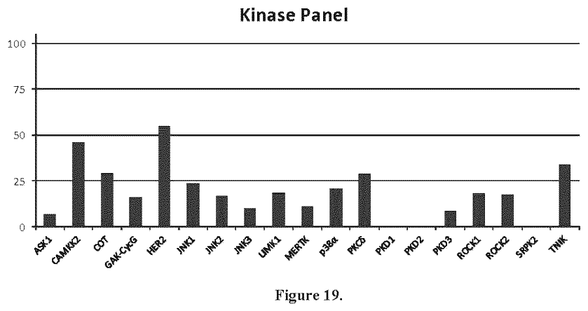

In FIG. 19, the Histogram (for 1/120 Final Dilution SACC) illustrates the inhibitory profile at a 1:120 dilution of the crude compound. Here, larger bars still signify higher inhibition levels. Surprisingly, at this more diluted concentration, only five kinases, constituting 10%, exhibit inhibition more than 25%. These include CAMKK2, COT/MAP3K8/Tpl2, HER2, PKCd, and TNIK/MAP4K7.

These targets play a key role in the interface of inflammation and cancer. In addition, as per Reyland et al., 2016, PKCδ is a tumour promoter in mouse models of mammary gland and lung cancer, and increased PKCδ expression is a negative prognostic indicator in Her2+ and other subtypes of human breast cancer.

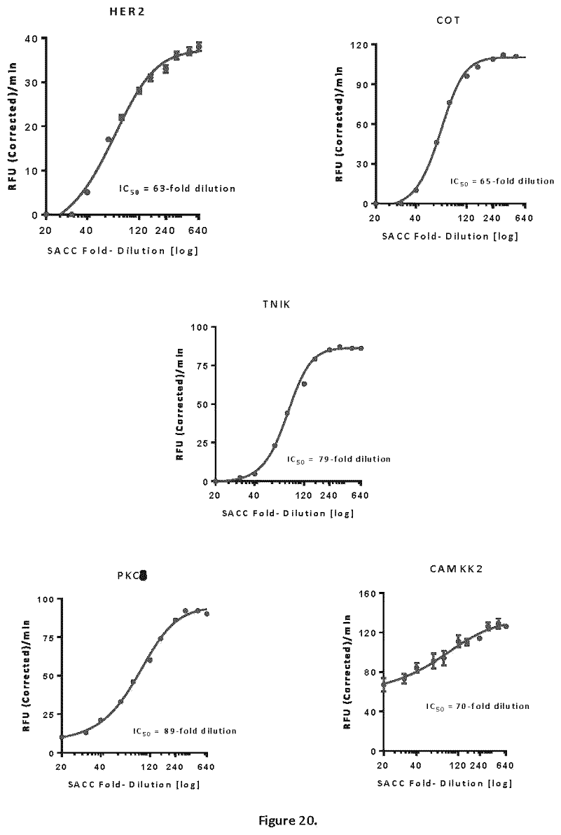

FIG. 20 provides a comprehensive view of Inhibitor Titrations for IC₅₀ Determination. The graph is arranged from left to right, representing a gradient from high to low concentrations of the crude compound. This titration analysis aims to determine the half-maximal inhibitory concentration (IC₅₀) of the compound across varying concentrations.

### Example 5: Cell Viability or MTT Assay

The effect of SACC fractions (peak 2, peak 3, peak 4 and peak 5) on viability of lung cells was determined by MTT assay.

### Procedure:

Cells were seeded in 96 wells plate at a density of 1×104 per well in 200 μL of respective complete culture medium containing 10% FBS and 1% antibiotics. After 70% confluency, cells were treated with the respective doses (indicated in the graph) of SACC and its fraction in complete culture medium for 24 h. After incubation for a specified time at 37° C. in a humidified incubator, dye MTT (5 mg/mL in PBS; diluted in 10 mL of serum-free medium) was added to each well and incubated for 2 h, after which the plate was centrifuged at 1,000 rpm for 5 min at 4° C. After careful removal of the medium, 0.1 mL buffered DMSO was added to each well. The absorbance was recorded on a microplate reader at the wavelength of 540 nm. The effect SACC and its fraction on cell growth inhibition was assessed as percent cell viability, where vehicle-treated cells were taken as 100% viable.

### Results:

Within all the SACC fractions, peaks 2, 3, and 4 exhibited the most pronounced inhibition of lung cancer cell growth, with inhibitory concentrations measured at 1.04 μg, 1.06 μg, and 1.02 μg, respectively. This suggests that these specific fractions contain potent bioactive components contributing to the observed anti-cancer effects. Overall, the results indicate that SACC is more therapeutically effective compared to individual fractions.

### Example 6: Cell Viability Test Using Trypan Blue Exclusion Method Against A549 Cell Line

Test sample: SACC composition
Standard: Doxorubicin drug

### Preparation of Test Solution:

For cytotoxicity studies, Test sample was considered as 100% stock and serial two-fold dilutions were prepared from 10% to 0.156% using F-12K plain media for treatment. 10 mM stock of Standard Doxorubicin was diluted from 100 μM to 1.56 μM using F-12K plain media for treatment.

### Cell Lines and Culture Medium:

A549 cells were cultured in F-12K medium with appropriate supplements in a humidified atmosphere of 5% CO₂ at 37° C. until confluent. The viability of the cells was checked using conventional hemocytometer. Appropriate cell density per ml of media was prepared and seeded in a 96 well plate and incubated for 24 h at 37° C. with 5% CO₂ to assess the cell viability.

### Procedure:

1. The cell count was adjusted to 5×10⁵ cells/ml using complete media. To each well of the 96 well microtiter plate, 100 μl of the diluted cell suspension (50,000 cells/well) was added and incubated for 24 h at 37° C. in 5% CO₂ atmosphere.
2. After 24 h, the cells were treated with various concentrations of test compound and incubated at 37° C. for 24 h in 5% CO₂ atmosphere.
3. After incubation the test solutions in the wells were discarded.
4. Cells were trypsinized and 20 μl of cell suspension was taken for viability counting.
5. To this 20 μl of trypan blue dye was added and mixed well.
6. The mixture was then loaded to haemocytometer at the end of the notch with the help of pipette.
7. Haemocytometer was kept under the inverted microscope and scored in each quadrant.
8. Cell counting wad done according to Neubauer's rules and noted. Percentage cell viability was calculated for the obtained data using below given formulae.

### Calculation:

Viable cells=Average viable cells*2*10³ cells/100 μl

% Viability=Viable cells/Total cells×100

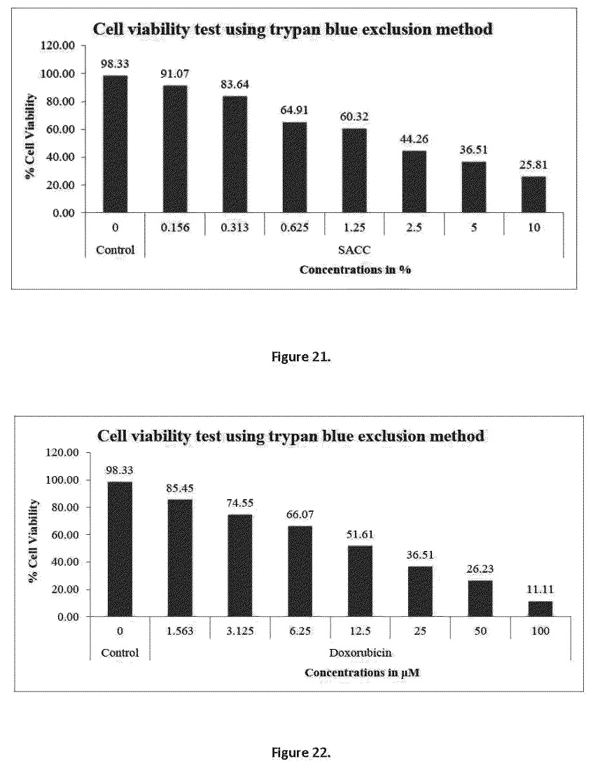

Table 1 demonstrates Cell Viability Assessment of Test Sample against A549 cell line and FIG. 21 shows the graph for cell viability after the treatment of Test Sample.

TABLE 1
Cell Viability Assessment of Test Sample against A549 cell line
A549

| | Conc. | Avg | Avg | | | | |
|---|---|---|---|---|---|---|---|
| Test | in | dead | viable | Dead | Viable | Total | % Cell |
| Sample | % | cells | cells | cells | cells | cells | Viability |
| Control | 0 | 1 | 59 | 2000 | 118000 | 120000 | 98.33 |
| SACC | 0.156 | 5 | 51 | 10000 | 102000 | 112000 | 91.07 |
| | 0.313 | 9 | 46 | 18000 | 92000 | 110000 | 83.64 |
| | 0.625 | 20 | 37 | 40000 | 74000 | 114000 | 64.91 |
| | 1.25 | 25 | 38 | 50000 | 76000 | 126000 | 60.32 |
| | 2.5 | 34 | 27 | 68000 | 54000 | 122000 | 44.26 |
| | 5 | 40 | 23 | 80000 | 46000 | 126000 | 36.51 |
| | 10 | 46 | 16 | 92000 | 32000 | 124000 | 25.81 |

Table 2 demonstrates cell Viability Assessment of Standard against A549 cell line and FIG. 22 shows the results of cell viability assessment of standard (Doxorubicin) against A549 cell line.

TABLE 2
Cell Viability Assessment of Standard against A549 cell line
A549

| | Conc. | Avg | Avg | | | | |
|---|---|---|---|---|---|---|---|
| Test | in | dead | viable | Dead | Viable | Total | % Cell |
| Sample | μM | cells | cells | cells | cells | cells | Viability |
| Control | 0 | 1 | 59 | 2000 | 118000 | 120000 | 98.33 |
| Doxo- | 1.563 | 8 | 47 | 16000 | 94000 | 110000 | 85.45 |
| rubicin | 3.125 | 14 | 41 | 28000 | 82000 | 110000 | 74.55 |
| | 6.25 | 19 | 37 | 38000 | 74000 | 112000 | 66.07 |
| | 12.5 | 30 | 32 | 60000 | 64000 | 124000 | 51.61 |
| | 25 | 40 | 23 | 80000 | 46000 | 126000 | 36.51 |
| | 50 | 45 | 16 | 90000 | 32000 | 122000 | 26.23 |
| | 100 | 56 | 7 | 112000 | 14000 | 126000 | 11.11 |

### Results:

In the experimental analysis, the test sample SACC and the standard (Doxorubicin) treatment demonstrated cell viability of 25.81% and 11.11%, respectively, when administered to A549 cells. The effect of SACC is demonstrated in FIG. 21 and of Doxorubicin is demonstrated in FIG. 22.

### Result Summary

| | | | Cell | |
|---|---|---|---|---|
| Cell Line | Test sample | Concentration | Viability | Method |
| A549 | SACC | 10% | 25.81% | Micro- |
| | Doxorubicin | 100 μM | 11.11% | scopy |

Test Sample SACC and Standard Doxorubicin showed 25.81% and 11.11% cell viability upon treatment against A549 cells respectively. These results indicate that the test sample, SACC, exhibited a cell viability higher than the standard treatment. Standard indicates potential toxicity or adverse effects on cell health.

### Example 7: Assessment of Cytotoxicity of Test Sample Against A549 Cell Line

### Preparation of Test Solution:

For cytotoxicity studies, Test sample was considered as 100% stock and serial two-fold dilutions were prepared from 10% to 0.156% using F-12K plain media for treatment. 10 mM stock of Standard Doxorubicin was diluted from 100 μM to 1.56 M using F-12K plain media for treatment.

### Cell Lines and Culture Medium:

A549 cells were cultured in F-12K medium with appropriate supplements in a humidified atmosphere of 5% CO₂ at 37° C. until confluent. The viability of the cells was checked using conventional hemocytometer. Appropriate cell density per ml of media was prepared and seeded in a 96 well plate and incubated for 24 h at 37° C. with 5% CO₂ to assess the cell viability.

### Procedure:

1. The cell count was adjusted to 5×105 cells/ml using complete media. To each well of the 96 well microtiter plate, 100 μl of the diluted cell suspension (50,000 cells/well) was added and incubated for 24 h at 37° C. in 5% CO₂ atmosphere.
2. After 24 h, the cells were treated with various concentrations of test compound and incubated at 37° C. for 24 h in 5% CO₂ atmosphere.
3. After incubation, the test solutions in the wells were discarded and 100 μl of MTT (5 mg/10 ml of MTT in PBS) was added to each well. The plates were incubated for 4 h at 37° C. in 5% CO₂ atmosphere.
4. The supernatants were removed and 100 μl of DMSO was added and the plates were gently shaken to solubilize the formed formazan. The absorbance was measured using a microplate reader at a wavelength of 590 nm.
5. The percentage growth inhibition was calculated using the following formula and concentration of test compound needed to inhibit cell growth by 50% (IC₅₀) values are generated from the dose response curves for cell line if the sample exhibits significant inhibition.

### Calculating Inhibition:

% Inhibition=(OD of sample−OD of Control/OD of Control)×100

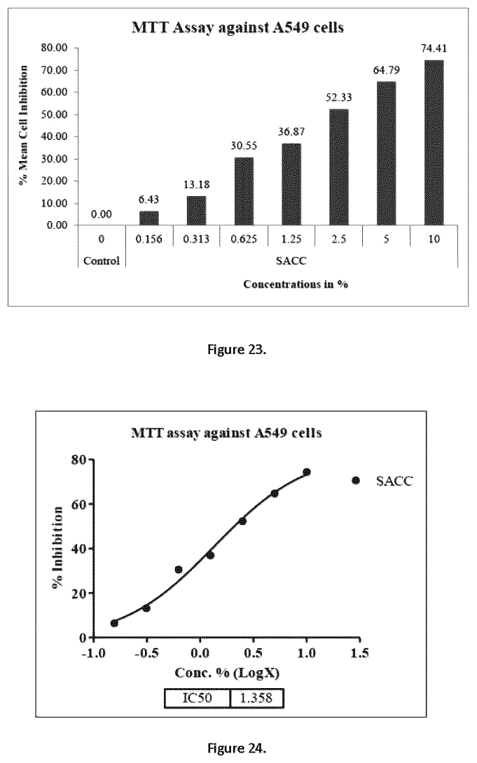

Table 1 demonstrates cytotoxicity Assessment of Test Sample against A549 cell line and FIGS. 23-24 show the bar and line graphs for cytotoxicity after the treatment of Test Sample.

TABLE 1
Cytotoxicity Assessment of Test Sample against A549 cell line
A549

| | | Absorbance @ 590 nm | | | | | | % Mean | | IC₅₀ |
|---|---|---|---|---|---|---|---|---|---|---|
| Test | Conc. | | % | | % | | % | Cell | | Value |
| Sample | in % | n = 1 | Inhibition | n = 2 | Inhibition | n = 3 | Inhibition | Inhibition | SD | in % |
| Control | 0 | 0.694 | 0.00 | 0.683 | 0.00 | 0.679 | 0.00 | 0.00 | 0.00 | 1.35 |
| SACC | 0.156 | 0.654 | 5.76 | 0.641 | 6.15 | 0.629 | 7.36 | 6.43 | 0.84 | |
| | 0.313 | 0.604 | 12.97 | 0.589 | 13.76 | 0.592 | 12.81 | 13.18 | 0.51 | |
| | 0.625 | 0.492 | 29.11 | 0.467 | 31.63 | 0.469 | 30.93 | 30.55 | 1.30 | |
| | 1.25 | 0.439 | 36.74 | 0.428 | 37.34 | 0.431 | 36.52 | 36.87 | 0.42 | |
| | 2.5 | 0.325 | 53.17 | 0.334 | 51.10 | 0.321 | 52.72 | 52.33 | 1.09 | |
| | 5 | 0.248 | 64.27 | 0.235 | 65.59 | 0.241 | 64.51 | 64.79 | 0.71 | |
| | 10 | 0.184 | 73.49 | 0.167 | 75.55 | 0.175 | 74.18 | 74.41 | 1.05 | |

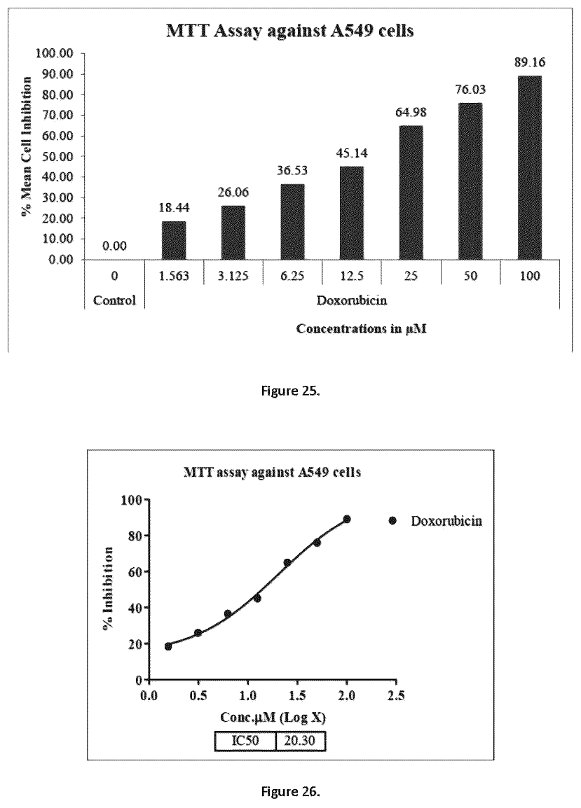

Table 2 demonstrates cytotoxicity Assessment of Standard (Doxorubicin) against A549 cell line and FIGS. 25-26 show the bar and line graphs for cytotoxicity after the treatment of Standard (Doxorubicin).

TABLE 2
Cytotoxicity Assessment of Standard against A549 cell line
A549

| | | | | | | | | | | IC₅₀ |
|---|---|---|---|---|---|---|---|---|---|---|
| | Conc. | Absorbance @ 590 nm | | | | | | % Mean | | Value |
| Test | in | | % | | % | | % | Cell | | in |
| Sample | μM | n = 1 | Inhibition | n = 2 | Inhibition | n = 3 | Inhibition | Inhibition | SD | μM |
| Control | 0 | 0.694 | 0.00 | 0.683 | 0.00 | 0.679 | 0.00 | 0.00 | 0.00 | 20.3 |
| Doxorubicin | 1.563 | 0.574 | 17.29 | 0.549 | 19.62 | 0.554 | 18.41 | 18.44 | 1.16 | |
| | 3.125 | 0.501 | 27.81 | 0.508 | 25.62 | 0.511 | 24.74 | 26.06 | 1.58 | |
| | 6.25 | 0.442 | 36.31 | 0.438 | 35.87 | 0.425 | 37.41 | 36.53 | 0.79 | |
| | 12.5 | 0.379 | 45.39 | 0.381 | 44.22 | 0.368 | 45.80 | 45.14 | 0.82 | |
| | 25 | 0.246 | 64.55 | 0.238 | 65.15 | 0.236 | 65.24 | 64.98 | 0.38 | |
| | 50 | 0.170 | 75.50 | 0.175 | 74.38 | 0.148 | 78.20 | 76.03 | 0.97 | |
| | 100 | 0.084 | 87.90 | 0.066 | 90.34 | 0.073 | 89.25 | 89.16 | 1.22 | |

### Results:

The test sample SACC and the standard drug Doxorubicin demonstrated inhibitory effects on A549 cells, as evidenced by their respective IC₅₀ values. Test Sample SACC and Standard Doxorubicin showed an IC₅₀ value of 1.35% (0.65 μM) and 20.3 μM inhibition in A549 cells respectively. FIGS. 23-26 demonstrate the comparison of cytotoxicity effect for SACC and Doxorubicin.

### Result Summary

| Cell Line | Test sample | IC₅₀ Value | Method |
|---|---|---|---|
| A549 | SACC | 1.35% | Colorimetric |
| | Doxorubicin | 20.3 μM | Assay |

The lower IC₅₀ value for SACC suggests higher potency in inhibiting the growth of A549 cells compared to the standard Doxorubicin. These findings highlight the promising anti-cancer properties of SACC as a potential therapeutic agent.

### Example 8: Therapeutic Activity of SACC Against Pancreatic Cancer

### 1. Therapeutic Efficacy of SACC in Human Pancreatic Cancer Cells (AsPC1) Ectopic Xenograft Mouse Model was Evaluated.

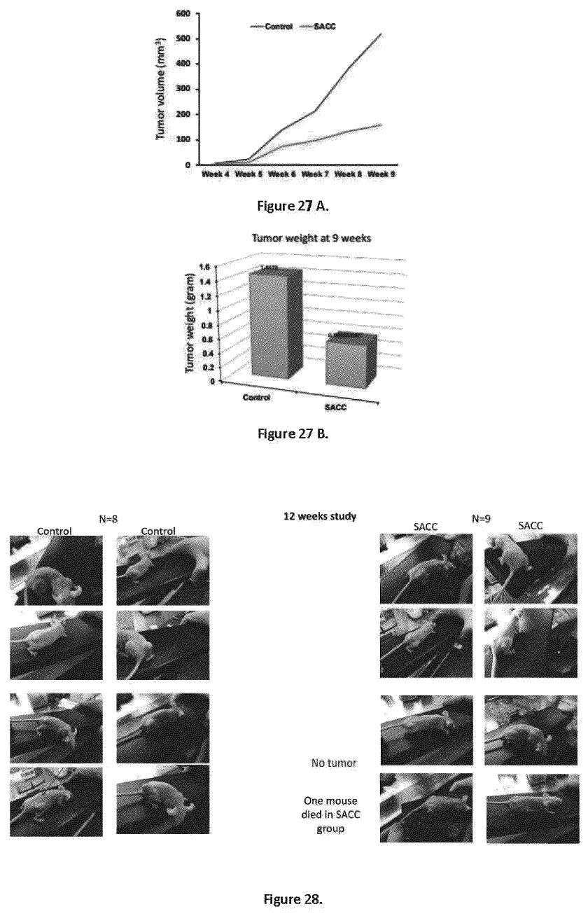

The results presented in FIGS. 27-30 demonstrate the inhibitory effect of SACC on the growth of pancreatic tumours in a xenograft mouse model of pancreatic cancer. In the athymic nude mice with xenograft tumours derived from AsPC1 cells, the intraperitoneal administration of 200 μl of SACC significantly inhibited tumour growth.

FIG. 27-A illustrates the tumour volume at indicated time points for both the control and SACC-administered groups. The values in the graphs represent the mean of 8 mice in the control group and 9 mice in the SACC-treated group. SACC was administered five days a week consecutively for 6 weeks. SACC administration, indicated by the significant reduction in tumour volume, highlights its potential as a promising anti-tumour agent.

FIG. 27 B shows the tumour weight of mice in both the control and SACC groups at 9 weeks. The values in the graph represent the mean of 8 mice in the control group and 9 mice in the SACC treatment group. The graph demonstrates a substantial decrease in tumour weight in the SACC-treated group. This further supports the anti-tumour efficacy of SACC in inhibiting the growth of pancreatic tumours.

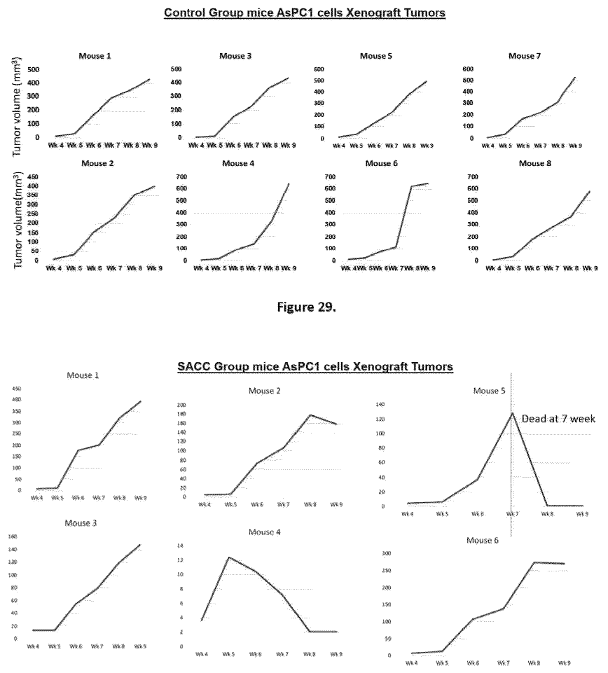

In FIG. 29, the line graph illustrates the growth pattern of AsPC1 cells Xenograft Tumours in the Control group mice. The graph provides a visual representation of tumour development over time, offering insights into the dynamics and progression of tumours in the absence of SACC treatment.

In FIG. 30, the line graph portrays the growth pattern of AsPC1 cells Xenograft Tumours in the SACC-treated group mice. This graph visually represents the impact of SACC administration on the growth curve of tumours over the experimental period.

Overall, the consistent reduction in tumour volume and weight in the SACC-treated group compared to the control group suggests a significant inhibitory effect of SACC on pancreatic tumour growth in this xenograft mouse model. The conclusion drawn from these findings is that SACC exhibits potential as a chemotherapeutic agent against pancreatic cancer.

### 2. SACC Inhibits the Growth of Pancreatic Cancer Stem Cells Xenograft Tumours in SCID Mice.

Effect of SACC on pancreatic cancer stem cells xenograft in SCID mice has been investigated wherein, a total of eight mice were used in the study. Briefly, 100% viable 2000 cells in 100 μl volume (1:1 ratio of Culture media+Matrigel) were subcutaneously injected in SCID mice. Two weeks later, SACC (250 μl) was administered intraperitoneally 5 days in a week for two weeks. All of the mice were sacrificed when control mice tumours reach a targeted volume 1500 mm³.

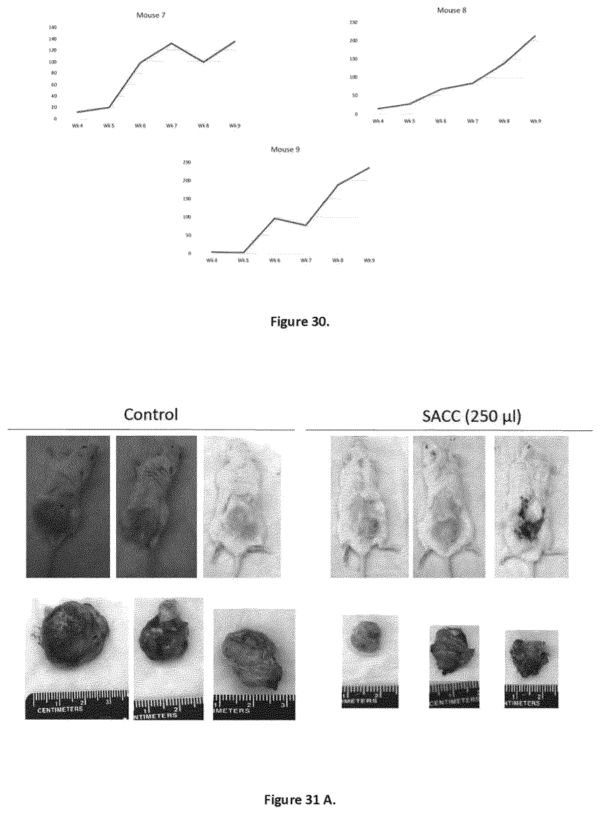

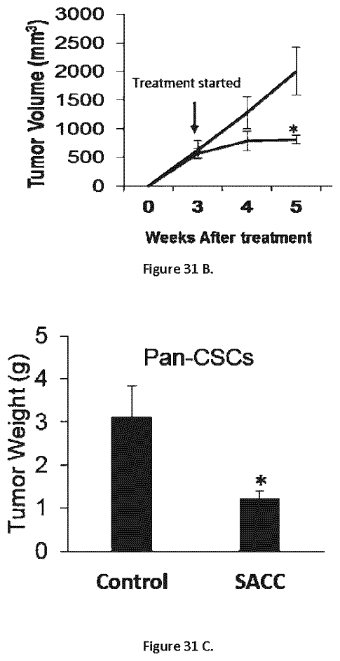

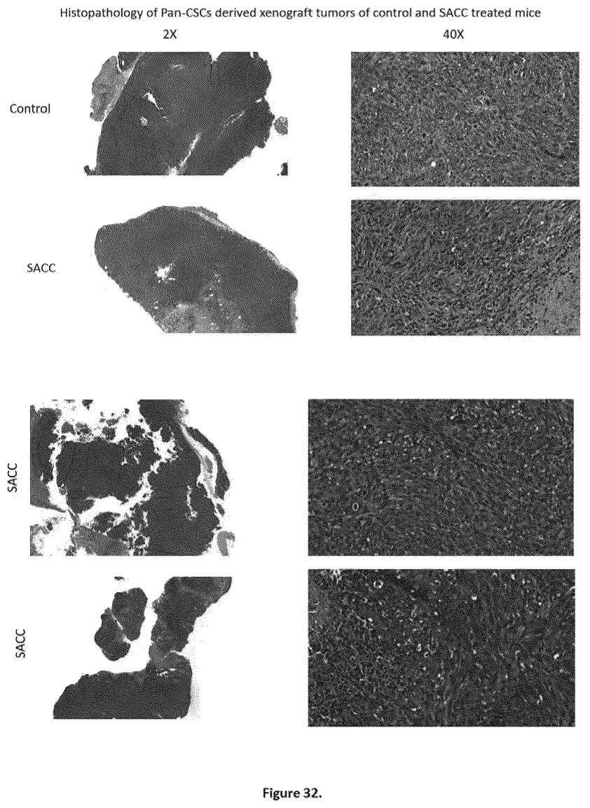

FIG. 31A presents images of mice bearing xenograft tumours in both the control and SACC-treated groups. The lower panel images display removed xenograft tumours from both sets of mice, allowing a visual comparison of tumour characteristics between the control and SACC-treated groups.

FIG. 31 B depicts the tumour volume of xenograft tumours, this graph illustrates the changes in tumour size over the indicated weeks for both the control and SACC-treated groups. The graph provides quantitative data on the impact of SACC treatment on tumour volume compared to the control group.

FIG. 31 C shows the removed xenograft tumour weights of mice in both the control and SACC-treated groups at week 5. The comparison of tumour weights presents insights into the inhibitory effect of SACC on tumour growth, providing additional support to the findings in FIG. 31B.

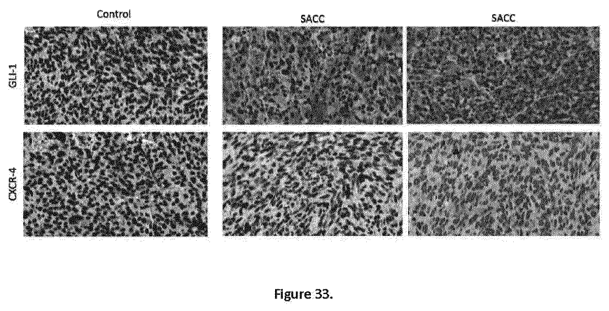

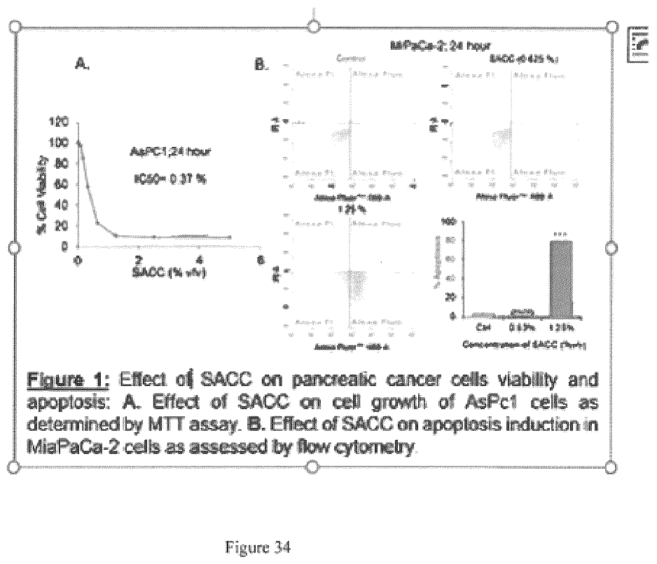

FIG. 33 explores the impact of SACC on the expression of transcription factors GLI-1 and chemokine receptor CXCR4. The representative Immunohistochemistry images display the expression levels of GLI-1 and CXCR4 in control and SACC xenograft tumours. The results indicate that SACC partially inhibits both GLI-1 and CXCR4, suggesting potential anti-tumour effects.

### Example 9: Molecular Mechanisms of SACC to Inhibit the Growth of Pancreatic Cancer

### Investigations on the Effect of SACC on MiaPaCa-2 and AsPc-1 Cells Through MTT Assay:

The anticancer potency of SACC was investigated in pancreatic cancer cells, i.e., MiaPaCa-2 and AsPc1, by using previously described MTT method [1]. The MiaPaCa-2 and AsPc-1 cells maintained in DMEM and RPMI-1640 medium, respectively, were seeded in 96-well plate at a seeding density of 5×10³ cells/well and left overnight at 5% CO₂ and at 37° C. for adherence. After 24 h, varying concentrations (0, 0.625% and 1.25%, v/v) of SACC were added to these adhered MiaPaCa-2 and AsPc-1 cells and incubated for further 24 h in similar growth conditions. Following the incubation, 0.2 mL of MTT (20% v/v, prepared either in DMEM or RPMI-1640 medium) was added to these cells, incubated for 4 h at 37° C. in dark and the generation of formazan crystals was assessed after solubilizing them into 0.15 mL DMSO by putting the plate on shaker and the plate was read at 570 nm.

### Immunoblotting

The MiaPaCa-2 and AsPc-1 cells were seeded in 100 mm culture dishes at a seeding density of 1.25×10⁶ and incubated at 37° C. under 5% CO₂. After 70% confluency, the MiaPaCa-2 and AsPc1 cells were treated with 0.625% and 1.25% (v/v) SACC for 24 h, while untreated cells were designated as control for each cell type. After 24 h of treatment, the cells were lysed in ice-cold RIPA buffer (containing protease and phosphatase inhibitors). The lysates were then centrifuged at 14,000×g for 15 min at 4° C. followed by the protein quantitation using the bicinchoninic acid (BCA) method. Twenty microgram protein from untreated as well as treated cell lysates were denatured at 95° C. for 7 min in sample buffer containing p3-mercaptoethanol and resolved on 10% gel at constant voltage of 75V. The resolved proteins were transferred on PVDF membrane using Bio rad's TransBlot® Turbo™ Transfer System (Cat No. #1704150EDU). After the transfer, membranes were blocked either with 5% non-fat milk or 5% bovine serum albumin (prepared in 1×TBST) for 2 h at 20 rpm on a shaker and then incubated overnight at 4° C. with primary antibodies against target proteins i.e., HER2/ErbB2 (Cell Signaling Technology; Cat. No. #4290S; Dil. 1:1000), pHER2, Dil. 1:1000), p53 (Santa Cruz Biotechnology, Dil. 1:3000); p21 (abcam; Cat No. #ab18209; Dil. 1:1000), Bcl2 (Santa Cruz Biotechnology; Cat No. #Sc-7382; Dil. 1:1000); Bax (Santa Cruz Biotechnology; Cat. No. #sc-65582; Dil. 1:1000), PARP, Dil. 1:1000), and p3-actin (Santa Cruz Biotechnology; Cat No. #sc-47778; Dil. 1:2000). After overnight incubation with primary antibodies, membrane was washed thrice with 1×TBST at 60-70 rpm to remove unbound antibodies followed by incubation either with anti-mouse or anti-rabbit HRP-conjugated antibodies for 1 h at 20 rpm. The unbound secondary antibodies were washed with 1×TBST as described for primary antibodies. Protein bands were detected by using Immobilon Western Chemiluminescent HRP Substrate (Cat #WBKLS0500) which is based on high sensitivity detection of chemiluminescence arising from immunoreactive bands. All the antibodies used were validated by the produced bands of the expected molecular weight (kDa) for the target protein.

### Results and Discussion:

SACC has the ability to induce apoptosis, a programmed cell death, and inhibit the growth of MiaPaCa-2 and AsPc-1 cells. This implies that SACC may have potential anti-cancer properties by triggering cell death mechanisms and restricting the proliferation of these specific cancer cell lines. The observation of apoptosis induction and growth inhibition in these cells highlights the potential therapeutic efficacy of SACC in combating cancer.

There are distinct strategies to determine the antiproliferative effect of any therapeutic regimen against various cancer cells and MTT assay, used to assess the viability of the cells, stands among the most preferred and prime strategies [1]. In the same context, the effect of SACC treatment on the viability of two pancreatic cancer cell lines i.e., MiaPaCa-2 and AsPc-1 cells having mutated and WT p53, respectively is investigated. The findings from present cell viability assay demonstrated that irrespective of p53 mutation SACC significantly inhibited the growth of pancreatic cancer cells. The IC₅₀ of SACC in AsPC1 cells was observed 0.37% (v/v) which showed IC₅₀ (This experiment is continue) in MiaPaCa-2. Further, the effect of SACC on apoptosis of PanCa cells is evaluated. SACC (1.25% v/v) treatment showed a significant apoptosis induction in MiaPaCa-2 cells. These results provide the evidence of SACC potential to kill the pancreatic cancer cells via inducing apoptosis. FIG. 34 shows the effect of SACC on pancreatic cancer cells viability and apoptosis.

### a) SACC Targets HER-2 and p53 Signaling Pathways in Pancreatic Cancer Cells.

The human epidermal growth factor receptor-2 (HER-2), also known as c-Neu/ErbB2, has shown profound effect on the pathophysiology of various cancers, most importantly, the pancreatic cancer, however, recent studies have already warned that its role in other range of cancers cannot be denied [2-7]. Even the genetic defects in HER-2 have also been correlated with severity of cancers via enhanced HER-2 functionality [3], thereby, making it a prime therapeutic target in the management of various cancers. Moreover, HER-2, alone or in association with MUC4, has also been correlated with the compromised efficacy of the currently available chemotherapies, particularly resistance to gemcitabine [7,8]. On the contrary, the therapies targeting HER-2 have shown their potential to sensitize the gemcitabine resistant pancreatic cells [9]. In this context, a set of anti-HER-2 therapeutic regimens have been explored and among these, the inhibitors of the tyrosine kinase are considered the drug of choice for HER-2 positive cancer phenotypes [10]. Like other chemotherapeutic drugs, even the best studied HER-2 blocker, Lapatinib, has shown serious adverse events including cardiac toxicity [11].

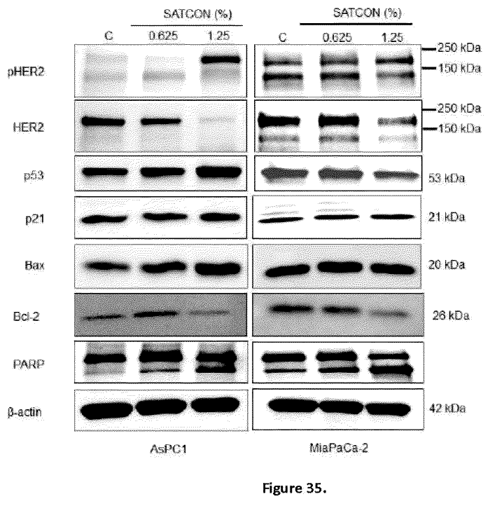

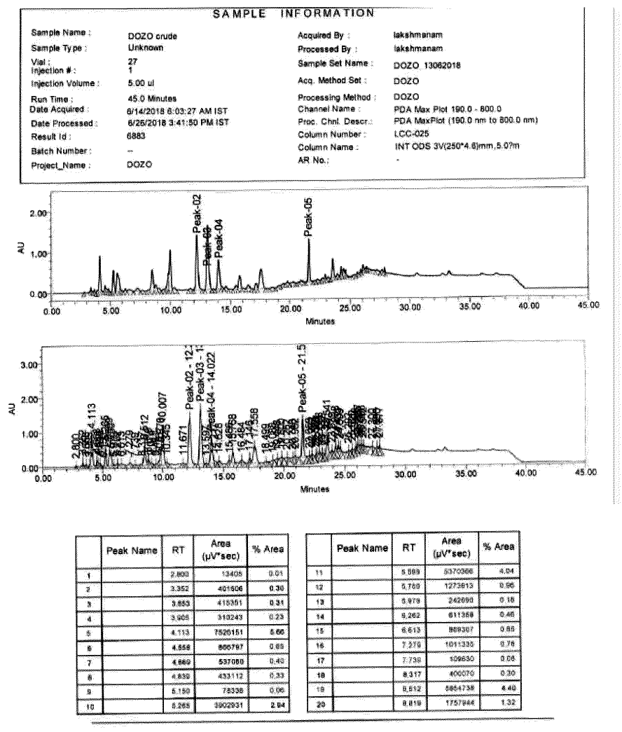

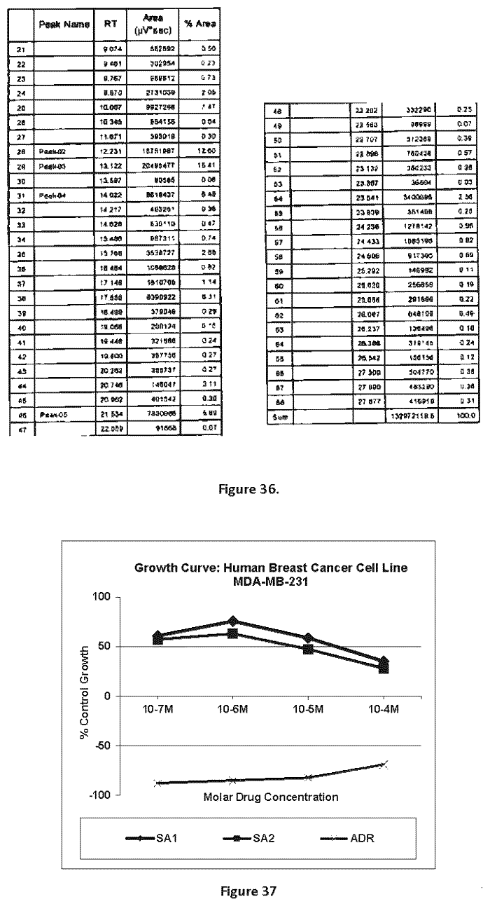

Therefore, considering these adverse events associated with different HER-2 targeting therapies, the inventor of the present disclosure explored if SACC could target HER-2 in pancreatic cancer cells. In present study, Western blot analysis is performed to investigate the effect of SACC on the expression of HER-2. The obtained results revealed that SACC down-regulates the protein expression of HER-2 in both the cell lines in a dose-dependent manner. Interestingly, SACC treatment enhances the phosphorylation of HER-2 in both AsPC-1 and MiaPaCa-2 cells at higher concentration (FIG. 35).

This effect was more in WT p53 expressing AsPC-1 cells compared to mutant p53 expressing MiaPaCa-2 cells. The present results are corroborating with a recently published study showed an enhanced phosphorylation of HER-2 in AsPC-1 with the treatment of chemotherapeutic drug gemcitabine [9].

### b) SACC Modulates the Expression of Apoptotic Proteins in Pancreatic Cancer Cells.

During apoptosis, cleavage of PARP has become a useful hallmark of this type of cell death. This cleavage is well studied and is generated by the caspases 3 and 7, proteases activated during apoptosis [12]. Similarly, in the present study, it is reported that the dose-dependent cytotoxicity of SACC was mediated by cleavage of PARP protein and subsequent apoptotic events in MiaPaCa-2 and AsPc1 cells. Most interestingly, SACC treatment resulted in a marked suppression of mutant p53 in MiaPaCa-2 cells, are known to possess mutant p53 (exon 3, 6, and, 7; R89W, R116W, R209W, R248W, C265T, C346T, C625T, and, C742T) [9,13] and the existence of this mutant p53 has also been associated with the gemcitabine resistance in pancreatic cancer cells, particularly MiaPaCa-2 [14].

Based on these previous observations, the downregulation of mutant p53 in MiaPaCa-2 cells in the current study signifies the antiproliferative efficacy of SACC. Interestingly, SACC treatment induced the expression of p53 in AsPC-1 cells [9]. These results suggest SACC induces apoptosis mediated cell death of pancreatic cells via targeting mutant p53, WT p53 and HER-2. Similarly, over-expression of various p53-targeted genes i.e., p21 has also been linked to the tumour suppression and growth inhibition/cellular arrest [15]. The findings from our study are very interesting as we reported an over-expression in p21 expression in AsPc-1 cells, which might be attributed to the induction of WT p53 by SACC treatment. Unlike AsPC-1 cells, the expression of p21 was unchanged in SACC treated MiaPaCa-2 cells. Moreover, the process of apoptosis is very complex and tightly regulated by coordination of distinct mediators including but not limited to Bcl-2 and Bcl-2-associated X, apoptosis regulator (BAX) [16]. BAX are known to trigger mitochondrial outer membrane permeabilization while Bcl-2 plays a negative role in the phenomenon of apoptosis [16]. In the same context, treatment with SACC markedly up-regulated the BAX/Bcl-2 ratio in both AsPc-1 and MiaPaCa-2 cells in a dose-dependent manner. These findings are clearly suggested that the SACC exerts antiproliferative potential against pancreatic cancer via targeting HER-2/p53/BAX-Bcl-2 axis.

### Conclusion

SACC induces apoptosis and inhibits the growth of pancreatic cancer cells irrespective of mutant p53. SACC inhibits the expression of HER-2 but increases its phosphorylation at higher dose. SACC induces the expression of wild type p53 protein but degrades the level of mutant p53 protein. SACC induces the expression of pro-apoptotic protein Bax and inhibits anti-apoptotic protein Bcl2. SACC induces the PARP protein cleavage.

Effect of SACC on the expression of WT p53 in CaPan-2 cells. These cells express WT p53. The status of p53 in AsPC-1 cells have reported to be a truncated form. However, many studies have reported that these cells express WT p53.

SACC composition is also studied on prostate xenograft and TNBC. SACC is also studied in CWR22Rv1 cell xenograft tumours in athymic nude mice.

Oral feeding of Satcon inhibits the CWR22v1 cell derived Xenograft tumours. In brief, a total of 8 athymic mice were used in the study. 4×106 cells were implanted subcutaneously into the dorsal surface of the flank of each mouse. Mice were divided into two groups. After one week, one group of mice was given SACC orally (300 ul 5 days in a week) for 3 weeks other group of mice received normal saline. Tumour volumes were recorded on 3^{rd} and 4^{th} week. All the mice of both the groups were sacrificed when control mice tumour volumes were reached approximately 2000 mm³. Blood was collected and tumours were excised and weight on electronic balance. At the end of Experiment, Tumour volume with SACC treated was 400 mm³ and tumour weight came down from 3.3 g to 1 gm. Significant activity is observed. It is strong inhibitor of TNBC xenograft tumour.

### Example 10: Therapeutic Activity of SA1 (Peak 5) and SA2 (Peak 4) Against Human Breast Cancer Cell Line MDA-MB-231

The therapeutic effect of SA (peak 5) and SA2 (peak 4) is evaluated against human lung cancer cell line MDA-MB-231 by calculating the percentage growth inhibition, drug concentration of test compound needed to inhibit cell growth by 50% (GI₅₀) values.

TABLE 1
represents the percentage of cell growth and drug concentration:

| | Human Breast Cancer Cell Line MDA-MB-231 | | | | | | | |
|---|---|---|---|---|---|---|---|---|
| | % Control Growth | | | | | | | |
| | Molar Drug Concentrations | | | | | | | |
| | Experiment 1 | | | | Experiment 2 | | | |
| | 10⁻⁷M | 10⁻⁶M | 10⁻⁵M | 10⁻⁴M | 10⁻⁷M | 10⁻⁶M | 10⁻⁵M | 10⁻⁴M |
| SA1 | 75.4 | 90.3 | 75.2 | 43.7 | 50.9 | 69.9 | 44.0 | 27.1 |
| (peak | | | | | | | | |
| 5) | | | | | | | | |
| SA2 | 81.0 | 68.2 | 45.5 | 50.1 | 42.6 | 59.7 | 42.0 | 18.1 |
| (peak | | | | | | | | |
| 4) | | | | | | | | |
| ADR | −82.8 | −83.1 | −80.5 | −68.3 | −91.0 | −86.2 | −83.4 | −69.6 |
| | Experiment 3 | | | | Average Values | | | |
| | 10⁻⁷M | 10⁻⁶M | 10⁻⁵M | 10⁻⁴M | 10⁻⁷M | 10⁻⁶M | 10⁻⁵M | 10⁻⁴M |
| SA1 | 56.0 | 66.1 | 56.4 | 34.3 | 60.8 | 75.4 | 58.6 | 35.0 |
| (peak | | | | | | | | |
| 5) | | | | | | | | |
| SA2 | 47.3 | 60.5 | 54.1 | 15.5 | 57.0 | 62.8 | 47.2 | 27.9 |
| (peak | | | | | | | | |
| 4) | | | | | | | | |
| ADR | −89.6 | −85.8 | −83.3 | −69.4 | −87.8 | −85.0 | −82.4 | −69.1 |

TABLE 2
represents the effect of SA1 (peak 5) and SA2 (peak 4) on cell
viability and tumour cell growth inhibition:

| | Molar drug concentrations | | |
|---|---|---|---|
| | (μg/ml) calculated from graph | | |
| MDA-MB-231 | LC₅₀ | TGI | GI₅₀* |
| SA1 (peak 5) | NE | 4.03 | 0.0000196 |
| SA2 (peak 4) | NE | 0.1727476 | 2.3811110 |
| ADR | 0.6191008 | <E−07 | <E−07 |

*GI50 value of ≤10⁻⁶ molar (i.e. 1 μmolar) or ≤10 μg/ml is considered to demonstrate activity in case of pure compounds. For extracts, GI₅₀ value ≤20 μg/ml is considered to demonstrate activity.
*These test values under GI₅₀ column indicates activity.
LC₅₀ = Concentration of drug causing 50% cell kill
GI₅₀ = Concentration of drug causing 50% inhibition of cell growth
TGI = Concentration of drug causing total inhibition of cell growth
ADR = Adriamycin, Positive control compound
NE = Non-evaluable data. Experiment needs to be repeated using different set of drug concentrations.
Erratic data = Erratic data can result due to less solubility of the compound.

### Results:

The test samples SA1 (peak 5) and SA2 (peak 4) demonstrated inhibitory effects on MDA-MB-231 cells, as evidenced by their respective GI₅₀ and TGI values. FIG. 37 demonstrates inhibition of cell growth for SA1 (peak 5), SA2 (peak 4), and Adriamycin.

### Example 11: Therapeutic Activity of SACC Against Human Breast Cancer Cell Line MDA-MB-231

The therapeutic effect of SACC is evaluated against human lung cancer cell lines MDA-MB-231 by calculating the percentage growth inhibition, drug concentration of test compound needed to inhibit cell growth by 50% (GI₅₀) values.

TABLE 1
represents the percentage of cell growth and drug concentration:

| | Human Breast Cancer Cell Line MDA-MB-231 | | | | | | | |
|---|---|---|---|---|---|---|---|---|
| | % Control Growth | | | | | | | |
| | Drug Concentrations (μg/ml) | | | | | | | |
| | Experiment 1 | | | | Experiment 2 | | | |
| | 10 | 20 | 40 | 80 | 10 | 20 | 40 | 80 |
| SACCRT | −26.6 | −52.5 | −59.7 | −58.2 | −30.6 | −56.9 | −64.4 | −59.9 |
| SACCD/RT | −40.4 | −54.3 | −68.2 | −59.8 | −57.4 | −59.6 | −69.2 | −63.4 |
| A6/2023 | −16.3 | −47.5 | −58.8 | −56.2 | −33.1 | −47.1 | −58.7 | −62.4 |
| ADR | −90.5 | −85.1 | −80.9 | −75.2 | −89.3 | −84.8 | −82.5 | −78.1 |
| | Experiment 3 | | | | Average Values | | | |
| | 10 | 20 | 40 | 80 | 10 | 20 | 40 | 80 |
| SACCRT | −44.5 | −58.8 | −64.3 | −58.1 | −33.9 | −56.1 | −62.8 | −58.8 |
| SACCD/RT | −57.1 | −60.8 | −68.2 | −49.7 | −51.6 | −58.2 | −68.5 | −57.6 |
| A6/2023 | −42.4 | −55.6 | −66.6 | −57.7 | −30.6 | −50.1 | −61.4 | −58.8 |
| ADR | −86.3 | −79.3 | −80.2 | −71.2 | −88.7 | −83.1 | −81.2 | −74.8 |

SACCRT: SACC stored for 6 months at 20° C.
SACCD/RT: Fresh sample of SACC
A6/2023: SACC stored for 6 months at 6° C.

TABLE 2
represents the effect of SACC on cell viability
and tumour cell growth inhibition:

| | Drug concentrations | | |
|---|---|---|---|
| | (μg/ml) calculated from graph | | |
| MDA-MB-231 | LC₅₀ | TGI | GI₅₀* |
| SACCRT | 26.3 | <10 | <10 |
| SACCD/RT | <10 | <10 | <10 |
| A6/2023 | 36.9 | <10 | <10 |
| ADR | <10 | <10 | <10 |

*GI50 value of ≤10⁻⁶ molar (i.e. 1 μmolar) or ≤10 μg/ml is considered to demonstrate activity in case of pure compounds. For extracts, GI₅₀ value ≤20 μg/ml is considered to demonstrate activity.
*These test values under GI₅₀ column indicates activity.

### Results:

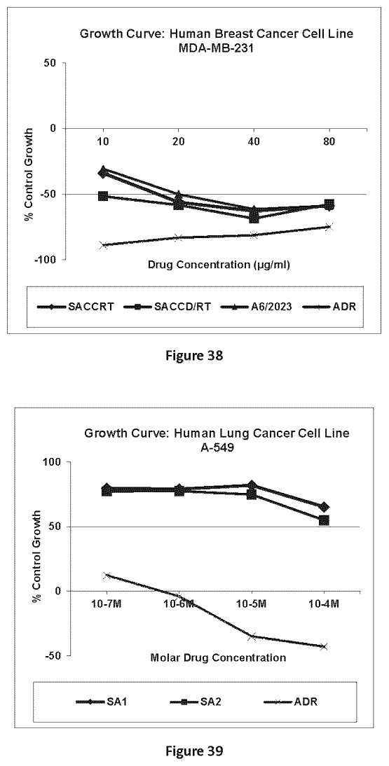

The test sample SACC and the control drug Adriamycin demonstrated inhibitory effects on MDA-MB-231 cells, as evidenced by their respective IC₅₀, GI₅₀, and TGI values. FIG. 38 demonstrates inhibition of cell growth for SACC and Adriamycin.

### Example 12: Therapeutic Activity of SA1 (Peak 5) and SA2 (Peak 4) Against Human Lung Cancer Cell Line A-549

The therapeutic effect of SA1 (peak 5) and SA2 (peak 4) is evaluated against human lung cancer cell line A-549 by calculating the percentage growth inhibition, drug concentration of test compound needed to inhibit cell growth by 50% (GI₅₀) values.

TABLE 1
represents the percentage of cell growth and drug concentration:

| | Human Lung Cancer Cell Line A-549 | | | | | | | |
|---|---|---|---|---|---|---|---|---|
| | % Control Growth | | | | | | | |
| | Molar Drug Concentrations | | | | | | | |
| | Experiment 1 | | | | Experiment 2 | | | |
| | 10⁻⁷M | 10⁻⁶M | 10⁻⁵M | 10⁻⁴M | 10⁻⁷M | 10⁻⁶M | 10⁻⁵M | 10⁻⁴M |
| SA1 | 80.8 | 83.6 | 86.4 | 64.0 | 80.6 | 77.7 | 78.3 | 65.1 |
| (peak | | | | | | | | |
| 5) | | | | | | | | |
| SA2 | 80.0 | 74.0 | 81.5 | 54.4 | 76.9 | 80.1 | 72.0 | 51.7 |
| (peak | | | | | | | | |
| 4) | | | | | | | | |
| ADR | 53.2 | 10.2 | −30.6 | 36.5 | 15.9 | −11.3 | −33.8 | 44.4 |
| | Experiment 3 | | | | Average Values | | | |
| | 10⁻⁷M | 10⁻⁶M | 10⁻⁵M | 10⁻⁴M | 10⁻⁷M | 10⁻⁶M | 10⁻⁵M | 10⁻⁴M |
| SA1 | 77.6 | 75.9 | 81.1 | 66.6 | 79.7 | 79.1 | 81.9 | 65.2 |
| (peak | | | | | | | | |
| 5) | | | | | | | | |
| SA2 | 74.9 | 78.2 | 70.9 | 58.6 | 77.3 | 77.4 | 74.8 | 54.9 |
| (peak | | | | | | | | |
| 4) | | | | | | | | |
| ADR | −32.0 | −11.0 | −40.3 | −48.1 | 12.4 | −4.0 | −34.9 | −43.0 |

TABLE 2
represents the effect of SA1 (peak 5) and SA2 (peak 4)
on cell viability and tumour cell growth inhibition:

| | Molar drug concentrations | | |
|---|---|---|---|
| | (μg/ml) calculated from graph | | |
| A-549 | LC₅₀ | TGI | GI₅₀* |
| SA1 (peak 5) | NE | NE | >E−04 |
| SA2 (peak 4) | NE | NE | 0.0033950 |
| ADR | 0.0001431 | 4.1431126 | 1.1994766* |

*GI50 value of ≤10⁻⁶ molar (i.e. 1 μmolar) or ≤10 μg/ml is considered to demonstrate activity in case of
pure compounds. For extracts, GI₅₀ value ≤20 μg/ml is considered to demonstrate activity.
*These test values under GI₅₀ column indicates activity.
NE = Non-evaluable data. Experiment needs to be repeated using different set of drug concentrations.
Erratic data = Erratic data can result due to less solubility of the compound.

### Results:

The test sample SA2 (peak 4) and the control drug Adriamycin both exhibited inhibitory effects on A-549. The inhibitory effect of SA2 is illustrated through its GI₅₀ values while Adriamycin's inhibition is supported by its respective LC₅₀, GI₅₀, and TGI values. FIG. 39 demonstrates inhibition of cell growth for SA1 (peak 5), SA2 (peak 4), and Adriamycin.

### Example 13: Therapeutic Activity of SACC Against Human Lung Cancer Cell Line A-549

The therapeutic effect of SACC is evaluated against human lung cancer cell line A-549 by calculating the percentage growth inhibition, drug concentration of test compound needed to inhibit cell growth by 50% (GI₅₀) values.

TABLE 1
represents the percentage of cell growth and drug concentration:

| | Human Lung Cancer Cell Line A-549 | | | | | | | | | | | | | | | |
|---|---|---|---|---|---|---|---|---|---|---|---|---|---|---|---|---|
| | % Control Growth | | | | | | | | | | | | | | | |
| | Drug Concentrations (μg/ml) | | | | | | | | | | | | | | | |
| | Experiment 1 | | | | Experiment 2 | | | | Experiment 3 | | | | Average Values | | | |
| | 10 | 20 | 40 | 80 | 10 | 20 | 40 | 80 | 10 | 20 | 40 | 80 | 10 | 20 | 40 | 80 |
| SACCRT | 32.3 | 7.8 | −72.5 | −79.6 | 32.3 | 5.2 | −75.7 | −79.9 | 31.3 | 8.5 | −75.9 | −78.4 | 32.0 | 7.2 | −74.7 | −79.3 |
| SACCD/RT | 32.8 | −11.9 | −83.0 | −75.1 | 28.7 | −19.8 | −85.0 | −75.6 | 29.5 | −28.9 | −86.0 | −73.9 | 30.3 | −20.2 | −84.7 | −74.9 |
| A6/2023 | 47.0 | 23.7 | −66.2 | −75.1 | 49.8 | 26.5 | −65.0 | −73.8 | 43.0 | 25.3 | −69.9 | −79.2 | 46.6 | 25.2 | −67.0 | −76.0 |
| ADR | −23.5 | −28.2 | −45.0 | −49.4 | −29.5 | −33.8 | −48.7 | −51.0 | −26.5 | −30.4 | −44.2 | −49.4 | −26.5 | −30.8 | −46.0 | −50.0 |

TABLE 2
represents the effect of SACC on cell viability
and tumour cell growth inhibition:

| | Drug concentrations (μg/ml) | | |
|---|---|---|---|
| | calculated from graph | | |
| A-549 | LC₅₀ | TGI | GI₅₀* |
| SACCRT | 50.9 | 19 | <10 |
| SACCD/RT | 46.9 | <10 | <10 |
| A6/2023 | 55.6 | 27 | <10 |
| ADR | 72 | <10 | <10 |

*GI50 value of ≤10⁻⁶ molar (i.e. 1 μmolar) or ≤10 μg/ml is considered to demonstrate activity in case of pure compounds. For extracts, GI50 value ≤20 μg/ml is considered to demonstrate activity.
*These test values under GI₅₀ column indicates activity.

### Results:

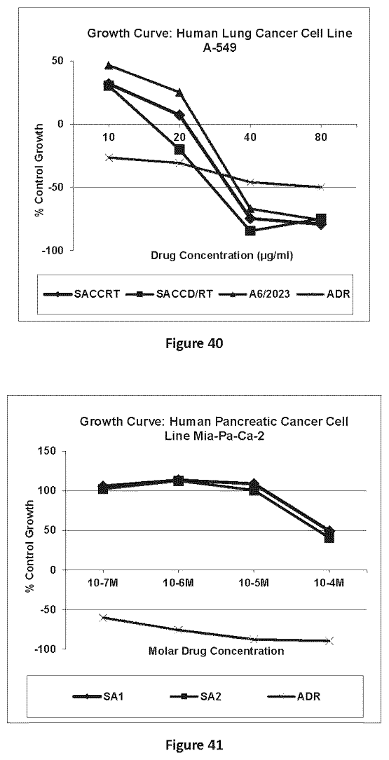

The test sample SACC and the control drug Adriamycin demonstrated inhibitory effects on A549 cells, as evidenced by their respective LC₅₀ and GI₅₀, and TGI values. Test Sample SACC and Adriamycin showed the LC₅₀ value of 46.9 and 72 respectively. FIG. 40 demonstrates inhibition of cell growth for SACC and Adriamycin.

### Example 14: Therapeutic Activity of SA1 (Peak 5) and SA2 (Peak 4) Against Human Pancreatic Cancer Cell Line Mia-Pa-Ca-2

The therapeutic effect of SA1 (peak 5) and SA2 (peak 4) is evaluated against human pancreatic cancer cell line Mia-Pa-Ca-2 by calculating the percentage growth inhibition, drug concentration of test compound needed to inhibit cell growth by 50% (GI₅₀) values.

TABLE 1
represents the percentage of cell growth and drug concentration:

| | Human Pancreatic Cancer Cell Line Mia-Pa-Ca-2 | | | | | | | |
|---|---|---|---|---|---|---|---|---|
| | % Control Growth | | | | | | | |
| | Molar Drug Concentrations | | | | | | | |
| | Experiment 1 | | | | Experiment 2 | | | |
| | 10⁻⁷M | 10⁻⁶M | 10⁻⁵M | 10⁻⁴M | 10⁻⁷M | 10⁻⁶M | 10⁻⁵M | 10⁻⁴M |
| SA1 | 108.1 | 111.0 | 97.0 | 30.0 | 95.5 | 116.4 | 112.7 | 63.5 |
| (peak 5) | | | | | | | | |
| SA2 | 95.4 | 114.3 | 76.9 | 40.0 | 111.8 | 115.6 | 116.3 | 36.6 |
| (peak 4) | | | | | | | | |
| ADR | −3.2 | −77.3 | −88.7 | −88.2 | −88.2 | −75.7 | −87.4 | −89.7 |
| | Experiment 3 | | | | Average Values | | | |
| | 10⁻⁷M | 10⁻⁶M | 10⁻⁵M | 10⁻⁴M | 10⁻⁷M | 10⁻⁶M | 10⁻⁵M | 10⁻⁴M |
| SA1 | 113.5 | 112.6 | 115.9 | 54.2 | 105.7 | 113.4 | 108.5 | 49.2 |
| (peak 5) | | | | | | | | |
| SA2 | 99.0 | 107.5 | 108.3 | 45.0 | 102.1 | 112.4 | 100.5 | 40.5 |
| (peak 4) | | | | | | | | |
| ADR | −89.3 | −74.5 | −87.3 | −91.0 | −60.2 | −75.8 | −87.8 | −89.6 |

TABLE 2
represents the effect of SA1 (peak 5) and SA2 (peak 4)
on cell viability and tumour cell growth inhibition:

| | Molar drug concentrations | | |
|---|---|---|---|
| | (μg/ml) calculated from graph | | |
| Mia-Pa-Ca-2 | LC₅₀ | TGI | GI₅₀* |
| SA1 (peak 5) | NE | 0.7992092 | 0.0010839 |
| SA2 (peak 4) | NE | 0.1053506 | 0.0003010 |
| ADR | 4.6306166 | 4.6904525 | 4.7510616* |

*GI50 value of ≤10⁻⁶ molar (i.e. 1 μmolar) or ≤10 μg/ml is considered to demonstrate activity in case of pure compounds. For extracts, GI₅₀ value ≤20 μg/ml is considered to demonstrate activity.
*These test values under GI₅₀ column indicate s activity.
NE = Non-evaluable data. Experiment needs to be repeated using different set of drug concentrations.

### Results:

The test samples SA1 (peak 5), SA2 (peak 4), and the control drug Adriamycin exhibited inhibitory effects on Mia-Pa-Ca-2. The inhibitory effects of SA1 and SA2 are illustrated through their its GI₅₀ and TGI values. And Adriamycin's inhibition is illustrated by its respective LC₅₀, GI₅₀, and TGI values. FIG. 41 demonstrates inhibition of cell growth for SA1 (peak 5), SA2 (peak 4), and Adriamycin.

### Example 15: Therapeutic Activity of SACC Against Human Pancreatic Cancer Cell Line Mia-Pa-Ca-2

The therapeutic effect of SACC is evaluated against human lung cancer cell line Mia-Pa-Ca-2 by calculating the percentage growth inhibition, drug concentration of test compound needed to inhibit cell growth by 50% (GI₅₀) values.

TABLE 1
represents the percentage of cell growth and drug concentration:

| | Human Pancreatic Cancer Cell Line Mia-Pa-Ca-2 | | | | | | | | | | | | | | | |
|---|---|---|---|---|---|---|---|---|---|---|---|---|---|---|---|---|
| | % Control Growth | | | | | | | | | | | | | | | |
| | Drug Concentrations (μg/ml) | | | | | | | | | | | | | | | |
| | Experiment 1 | | | | Experiment 2 | | | | Experiment 3 | | | | Average Values | | | |
| | 10 | 20 | 40 | 80 | 10 | 20 | 40 | 80 | 10 | 20 | 40 | 80 | 10 | 20 | 40 | 80 |
| SACCRT | −27.7 | 73.2 | −92.4 | −85.3 | −37.9 | −71.3 | −88.7 | −83.5 | 52.6 | −75.4 | −88.1 | −84.5 | −39.4 | −73.3 | −89.7 | −84.5 |
| SACCD/ | −28.5 | −85.2 | −91.5 | −83.1 | −46.3 | −63.8 | −88.3 | −82.1 | −61.9 | −84.2 | −88.8 | −82.4 | −45.5 | −77.7 | −89.5 | −82.5 |
| RT | | | | | | | | | | | | | | | | |
| A6/2023 | 20.4 | −51.6 | −86.4 | −82.6 | −10.9 | −48.4 | −83.4 | −87.4 | −19.0 | −48.7 | −78.4 | −86.2 | −3.2 | −49.6 | −82.7 | −85.4 |
| ADR | −88.7 | −91.3 | −91.4 | −84.1 | −89.2 | −90.4 | −92.3 | −87.5 | −91.2 | −91.5 | −91.0 | −82.3 | −89.7 | −91.1 | −91.5 | −84.7 |

TABLE 2
represents the effect of SACC on cell viability
and tumour cell growth inhibition:

| | Drug concentrations (μg/ml) | | |
|---|---|---|---|
| | calculated from graph | | |
| Mia-Pa-Ca-2 | LC₅₀ | TGI | GI₅₀* |
| SACCRT | <10 | <10 | <10 |
| SACCD/RT | <10 | <10 | <10 |
| A6/2023 | 32 | <10 | <10 |
| ADR | <10 | <10 | <10 |

*GI50 value of ≤10⁻⁶ molar (i.e. 1 μmolar) or ≤10 μg/ml is considered to demonstrate activity in case of pure compounds. For extracts, GI₅₀ value ≤20 μg/ml is considered to demonstrate activity.
*These test values under GI₅₀ column indicates activity.

### Results:

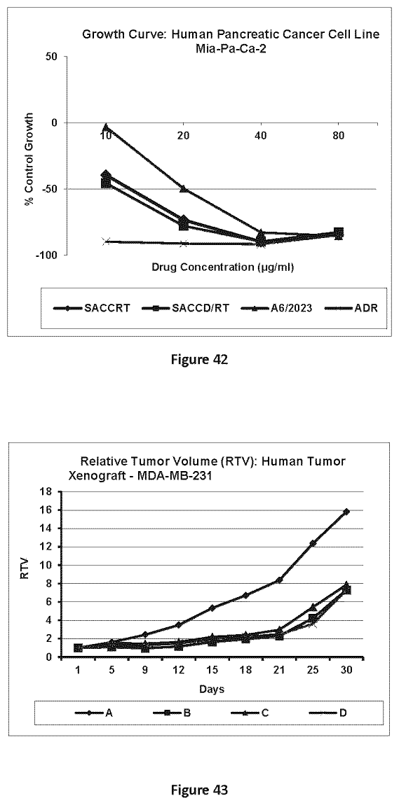

The test sample SACC and the control drug Adriamycin demonstrated inhibitory effects on Mia-Pa-Ca-2 cells, as evidenced by their respective LC₅₀ and GI₅₀, and TGI values. FIG. 42 demonstrates inhibition of cell growth for SACC and Adriamycin.

### Example 16: Assessing Therapeutic Efficacy of SACC Against Triple-Negative Breast Cancer Cell Line MDA-MB-231 in Human Tumour Xenograft MDA-MB-231 Models Through Tumour Volume and Relative Tumour Volume (RTV) Evaluation

The therapeutic effect of SACC is evaluated against triple-negative breast cancer cell line MDA-MB-231 by analysing tumour volume and relative tumour volume. The study involved the use of six mice. These mice were administrated treatment according to the following Groups:

| | |
|---|---|
| Group A | Control |
| Group B | Positive Control (ADR) is given in amount of 2.5 mg/kg |
| | via intravenous injection on day 1, day 5, day 9. |
| Group C | SACC is given in amount of 4 mL/Kg via oral |
| | administrationt wice a week for 4 weeks. |
| Group D | SACC is given in amount of 1.2 mL/Kg via oral |
| | administration daily 6 days/Week for 4 weeks. |

TABLE 1
Relative Tumour Volume in Group A, Group B, Group C, Group D

| | | RTV | | | |
|---|---|---|---|---|---|
| Weeks | Days | A | B | C | D |
| 0.00 | 1 | 1.00 | 1.00 | 1.00 | 1.00 |
| 0.71 | 5 | 1.61 | 1.06 | 1.50 | 1.21 |
| 1.29 | 9 | 2.43 | 0.95 | 1.45 | 1.27 |
| 1.71 | 12 | 3.48 | 1.14 | 1.63 | 1.48 |
| 2.14 | 15 | 5.32 | 1.63 | 2.18 | 1.91 |
| 2.57 | 18 | 6.71 | 1.93 | 2.40 | 2.22 |
| 3.00 | 21 | 8.35 | 2.29 | 2.96 | 2.45 |
| 3.57 | 25 | 12.36 | 4.22 | 5.43 | 3.61 |
| 4.29 | 30 | 15.81 | 7.30 | 7.86 | 7.22 |

*RTV = Relative Tumour Volume = Tumour Volume on day of measurement/Tumour Volume on day 1

### Results:

The test sample SACC and the control drug Adriamycin exhibited reduced tumour volume in human tumour xenograft MDA-MB-231 models, as indicated by their relative tumour volume. FIG. 43 demonstrates the decline in relative tumour volume for SACC and Adriamycin.

### Results:

The test sample SACC and the control drug Adriamycin exhibited reduced tumour volume in human tumour xenograft MDA-MB-231 models, as indicated by their T/C values achieved from RTV data. FIG. 44 demonstrates the decline in T/C values for SACC and Adriamycin.

TABLE 3
Percentage of survival in Group A, Group B, Group C, Group D

| | % Survival | | | | |
|---|---|---|---|---|---|
| Weeks | Days | A | B | C | D |
| 0.00 | 1 | 100 | 100 | 100 | 100 |
| 0.71 | 5 | 100 | 100 | 100 | 100 |
| 1.29 | 9 | 100 | 100 | 100 | 100 |
| 1.71 | 12 | 100 | 100 | 100 | 100 |
| 2.14 | 15 | 100 | 100 | 100 | 100 |
| 2.57 | 18 | 100 | 100 | 100 | 100 |
| 3.00 | 21 | 100 | 100 | 100 | 100 |
| 3.57 | 25 | 100 | 100 | 100 | 100 |
| 4.29 | 30 | 100 | 83 | 100 | 83 |

### Results:

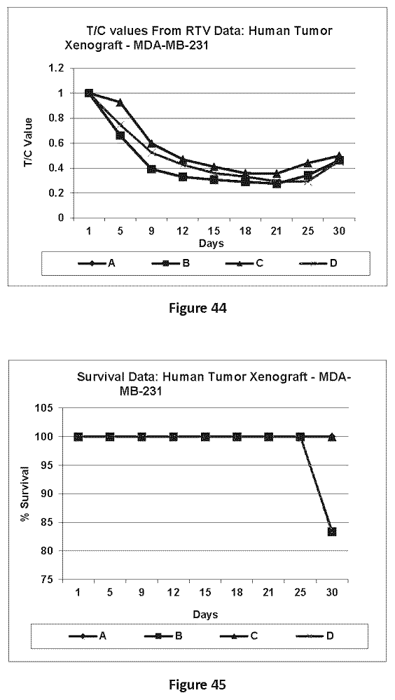

The test sample SACC and the control drug Adriamycin demonstrated good survival rate in human tumour xenograft MDA-MB-231 models, as indicated by their percentage of survival. FIG. 45 demonstrates the survival rate of 100% till 25 days for SACC and Adriamycin.

TABLE 4
Animal Body Weight data for
Group A, Group B, Group C, Group D

| | Animal Body Weight. (Grams) data | | | | |
|---|---|---|---|---|---|
| Weeks | Days | A | B | C | D |
| 0.0 | 1 | 21.7 | 20.9 | 22.5 | 23.2 |
| 0.7 | 5 | 21.8 | 19.9 | 22.2 | 22.8 |
| 1.3 | 9 | 22.1 | 20.3 | 21.9 | 23.1 |
| 1.7 | 12 | 21.8 | 20.1 | 21.9 | 23.1 |
| 2.1 | 15 | 20.6 | 19.5 | 22.0 | 23.3 |
| 2.6 | 18 | 22.2 | 20.4 | 21.9 | 22.9 |
| 3.0 | 21 | 21.6 | 20.0 | 21.4 | 22.9 |
| 3.6 | 25 | 21.8 | 20.2 | 21.8 | 22.9 |
| 4.3 | 30 | 22.0 | 21.6 | 22.7 | 23.3 |

*Mortality and weight loss ≥4 grams/mouse are considered to indicate toxicity.

### Results:

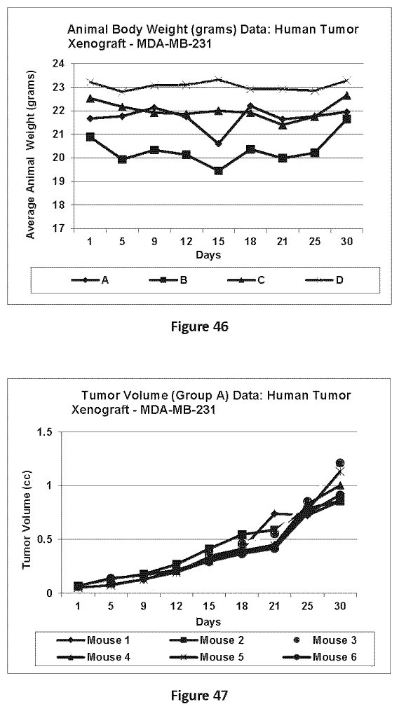

The test sample SACC and the control drug Adriamycin demonstrated minimal change in body weight in human tumour xenograft MDA-MB-231 models, as indicated by their average body weight. FIG. 46 demonstrates the graph of average animal body weight for SACC and Adriamycin.

TABLE 5
Tumour volume in Group A

| | | Tumor Volume Group A | | | | | |
|---|---|---|---|---|---|---|---|
| Week | Days | Mouse 1 | Mouse 2 | Mouse 3 | Mouse 4 | Mouse 5 | Mouse 6 |
| 0.00 | 1 | 0.06 | 0.07 | 0.06 | 0.07 | 0.05 | 0.07 |
| 0.71 | 5 | 0.09 | 0.09 | 0.10 | 0.13 | 0.07 | 0.14 |
| 1.29 | 9 | 0.12 | 0.18 | 0.16 | 0.18 | 0.13 | 0.17 |
| 1.71 | 12 | 0.22 | 0.27 | 0.23 | 0.21 | 0.19 | 0.22 |
| 2.14 | 15 | 0.35 | 0.41 | 0.36 | 0.32 | 0.30 | 0.29 |
| 2.57 | 18 | 0.41 | 0.54 | 0.46 | 0.39 | 0.39 | 0.37 |
| 3.00 | 21 | 0.74 | 0.59 | 0.55 | 0.45 | 0.44 | 0.42 |
| 3.57 | 25 | 0.72 | 0.79 | 0.85 | 0.83 | 0.78 | 0.74 |
| 4.29 | 30 | 0.86 | 0.86 | 1.21 | 1.00 | 1.13 | 0.91 |

This Table 5 and FIG. 47 represent the tumour volume in Control Group A.

TABLE 6
Tumour volume in Group B

| | | Tumor Volume Group B | | | | | |
|---|---|---|---|---|---|---|---|
| Week | Days | Mouse 1 | Mouse 2 | Mouse 3 | Mouse 4 | Mouse 5 | Mouse 6 |
| 0.00 | 1 | 0.06 | 0.05 | 0.06 | 0.05 | 0.06 | 0.06 |
| 0.71 | 5 | 0.05 | 0.07 | 0.06 | 0.06 | 0.05 | 0.06 |
| 1.29 | 9 | 0.05 | 0.08 | 0.04 | 0.04 | 0.06 | 0.05 |
| 1.71 | 12 | 0.06 | 0.10 | 0.05 | 0.05 | 0.06 | 0.07 |
| 2.14 | 15 | 0.07 | 0.16 | 0.07 | 0.06 | 0.08 | 0.11 |
| 2.57 | 18 | 0.07 | 0.21 | 0.07 | 0.07 | 0.07 | 0.16 |
| 3.00 | 21 | 0.05 | 0.23 | 0.08 | 0.11 | 0.13 | 0.17 |
| 3.57 | 25 | 0.08 | 0.31 | 0.33 | 0.24 | 0.17 | 0.29 |
| 4.29 | 30 | | 0.53 | 0.55 | 0.29 | 0.28 | 0.40 |

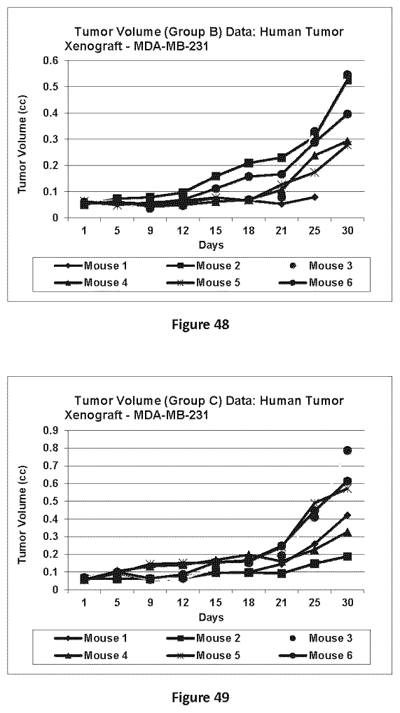

This Table 6 and FIG. 48 represent the tumour volume in Positive Control ADR (2.5 mg/Kg) Group B.

### Results:

The positive control drug Adriamycin demonstrated decreased tumour volume in human tumour xenograft MDA-MB-231 models.

TABLE 7
Tumour volume in Group C

| | | Tumor Volume Group C | | | | | |
|---|---|---|---|---|---|---|---|
| Week | Days | Mouse 1 | Mouse 2 | Mouse 3 | Mouse 4 | Mouse 5 | Mouse 6 |
| 0.00 | 1 | 0.07 | 0.06 | 0.07 | 0.06 | 0.05 | 0.06 |
| 0.71 | 5 | 0.09 | 0.06 | 0.10 | 0.10 | 0.09 | 0.10 |
| 1.29 | 9 | 0.06 | 0.06 | 0.06 | 0.13 | 0.14 | 0.06 |
| 1.71 | 12 | 0.06 | 0.08 | 0.06 | 0.14 | 0.15 | 0.09 |
| 2.14 | 15 | 0.09 | 0.10 | 0.12 | 0.17 | 0.15 | 0.16 |
| 2.57 | 18 | 0.10 | 0.10 | 0.15 | 0.20 | 0.16 | 0.17 |
| 3.00 | 21 | 0.14 | 0.09 | 0.19 | 0.16 | 0.24 | 0.25 |
| 3.57 | 25 | 0.26 | 0.15 | 0.41 | 0.23 | 0.49 | 0.45 |
| 4.29 | 30 | 0.42 | 0.19 | 0.79 | 0.33 | 0.57 | 0.61 |

This Table 7 and FIG. 49 represent the tumour volume in SACC (4 mL/Kg) Group C.

### Results:

The SACC, administrated at a dosage of 4 mL/Kg demonstrated reduction in tumour volume in human tumour xenograft MDA-MB-231 models.

TABLE 8
Tumour volume in Group D

| | | Tumor Volume Group D | | | | | |
|---|---|---|---|---|---|---|---|
| Week | Days | Mouse 1 | Mouse 2 | Mouse 3 | Mouse 4 | Mouse 5 | Mouse 6 |
| 0.00 | 1 | 0.06 | 0.05 | 0.06 | 0.05 | 0.06 | 0.06 |
| 0.71 | 5 | 0.05 | 0.07 | 0.06 | 0.06 | 0.05 | 0.06 |
| 1.29 | 9 | 0.05 | 0.08 | 0.04 | 0.04 | 0.06 | 0.05 |
| 1.71 | 12 | 0.06 | 0.10 | 0.05 | 0.05 | 0.06 | 0.07 |
| 2.14 | 15 | 0.07 | 0.16 | 0.07 | 0.06 | 0.08 | 0.11 |
| 2.57 | 18 | 0.07 | 0.21 | 0.07 | 0.07 | 0.07 | 0.16 |
| 3.00 | 21 | 0.05 | 0.23 | 0.08 | 0.11 | 0.13 | 0.17 |
| 3.57 | 25 | 0.08 | 0.31 | 0.33 | 0.24 | 0.17 | 0.29 |
| 4.29 | 30 | | 0.53 | 0.55 | 0.29 | 0.28 | 0.40 |

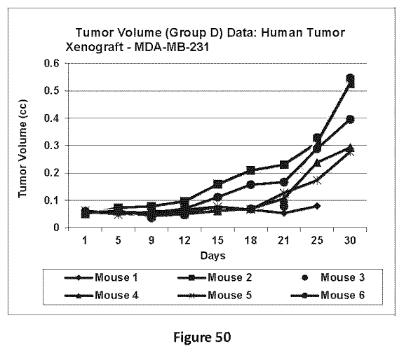

This Table 8 and FIG. 50 represent the tumour volume in SACC (4 mL/Kg) Group D.

### Results:

The SACC, administrated at a dosage of 1.2 mL/Kg, exhibited a significant reduction in tumour volume in human tumour xenograft MDA-MB-231 models.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

### REFERENCES

[1]R. Nabi, S. S. Alvi, A. Shah, C. P. Chaturvedi, D. Iqbal, S. Ahmad, M. S. Khan, Modulatory role of HMG-CoA reductase inhibitors and ezetimibe on LDL-AGEs-induced ROS generation and RAGE-associated signalling in HEK-293 Cells, Life Sci. 235 (2019) 116823.
[2]M. M. Moasser, The oncogene HER2: Its signaling and transforming functions and its role in human cancer pathogenesis, Oncogene. 26 (2007) 6469-6487.
[3]D. M. Hyman, S. A. Piha-Paul, H. Won, J. Rodon, C. Saura, G. I. Shapiro, D. Juric, D. I. Quinn, V. Moreno, B. Doger, I. A. Mayer, V. Boni, E. Calvo, S. Loi, A. C. Lockhart, J. P. Erinjeri, M. Scaltriti, G. A. Ulaner, J. Patel, J. Tang, H. Beer, S. Duygu Selcuklu, A. J. Hanrahan, N. Bouvier, M. Melcer, R. Murali, A. M. Schram, L. M. Smyth, K. Jhaveri, B. T. Li, A. Drilon, J. J. Harding, G. Iyer, B. S. Taylor, M. F. Berger, R. E. Cutler, F. Xu, A. Butturini, L. D. Eli, G. Mann, C. Farrell, A. S. Lalani, R. P. Bryce, C. L. Arteaga, F. Meric-Bernstam, J. Baselga, D. B. Solit, HER kinase inhibition in patients with HER2- and HER3-mutant cancers, Nature. 554 (2018) 189-194.
[4]A. Majumder, M. Sandhu, D. Banerji, V. Steri, A. Olshen, M. M. Moasser, The role of HER2 and HER3 in HER2-amplified cancers beyond breast cancers, Sci. Rep. 11 (2021) 9091.
[5]S. K. Joshi, J. M. Keck, C. A. Eide, D. Bottomly, E. Traer, J. W. Tyner, S. K. McWeeney, C. E. Tognon, B. J. Druker, ERBB2/HER2 mutations are transforming and therapeutically targetable in leukemia, Leukemia. 34 (2020) 2798-2804. https://doi.org/10.1038/s41375-020-0844-7.
[6]W. Wen, W. S. Chen, N. Xiao, R. Bender, A. Ghazalpour, Z. Tan, J. Swensen, S. Z. Millis, G. Basu, Z. Gatalica, M. F. Press, Mutations in the kinase domain of the HER2/ERBB2 gene identified in a wide variety of human cancers, J. Mol. Diagnostics. 17 (2015) 487-495.
[7]P. Chaturvedi, A. P. Singh, S. Chakraborty, S. C. Chauhan, S. Bafna, J. L. Meza, P. K. Singh, M. A. Hollingsworth, P. P. Mehta, S. K. Batra, MUC4 Mucin Interacts with and stabilizes the HER2 Oncoprotein in Human Pancreatic Cancer Cells, Cancer Res. 68 (2008) 2065-2070.
[8]S. Bafna, S. Kaur, N. Momi, S. K. Batra, Pancreatic cancer cells resistance to gemcitabine: the role of MUC4 mucin, Br. J. Cancer. 101 (2009) 1155-1161.
[9]H. Yao, W. Song, R. Cao, C. Ye, L. Zhang, H. Chen, J. Wang, Y. Shi, R. Li, Y. Li, X. Liu, X. Zhou, R. Shao, L. Li, An EGFR/HER2-targeted conjugate sensitizes gemcitabine-sensitive and resistant pancreatic cancer through different SMAD4-mediated mechanisms, Nat. Commun. 13 (2022) 5506. https://doi.org/10.1038/s41467-022-33037-x.
[10]I. Schlam, S. M. Swain, HER2-positive breast cancer and tyrosine kinase inhibitors: the time is now, Npj Breast Cancer. 7 (2021) 1-12. https://doi.org/10.1038/s41523-021-00265-1.
[11]B. Moy, P. E. Goss, Lapatinib-Associated Toxicity and Practical Management Recommendations, Oncologist. 12 (2007) 756-765. https://doi.org/10.1634/theoncologist.12-7-756.
[12]S. Gobeil, C. C. Boucher, D. Nadeau, G. G. Poirier, Characterization of the necrotic cleavage of poly (ADP-ribose) polymerase (PARP-1): Implication of lysosomal proteases, Cell Death Differ. 8 (2001) 588-594. https://doi.org/10.1038/sj.cdd.4400851.
[13]R. Gradiz, H. C. Silva, L. Carvalho, M. F. Botelho, A. Mota-Pinto, MIA PaCa-2 and PANC-1-Pancreas ductal adenocarcinoma cell lines with neuroendocrine differentiation and somatostatin receptors, Sci. Rep. 6 (2016) 1-14. https://doi.org/10.1038/srep21648.
[14]C. Fiorini, M. Cordani, C. Padroni, G. Blandino, S. Di Agostino, M. Donadelli, Mutant p53 stimulates chemoresistance of pancreatic adenocarcinoma cells to gemcitabine, Biochim. Biophys. Acta-Mol. Cell Res. 1853 (2015) 89-100.
[15]A. M. Boutelle, L. D. Attardi, p53 and Tumour Suppression: It Takes a Network, Trends Cell Biol. 31 (2021) 298-310.
[16]T. Yamazaki, L. Galluzzi, BAX and BAK dynamics control mitochondrial DNA release during apoptosis, Cell Death Differ. 29 (2022) 1296-1298. https://doi.org/10.1038/s41418-022-00985-2

1. A pharmaceutical composition comprising the following compounds:
(i) 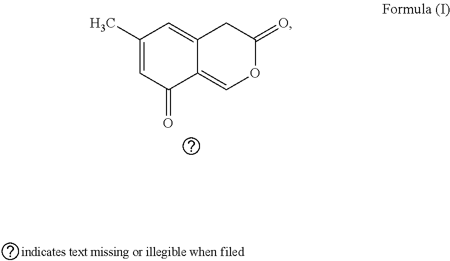
(ii) 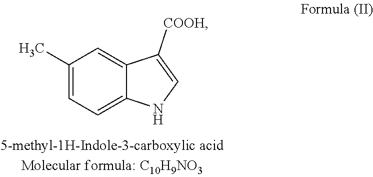
(iii) 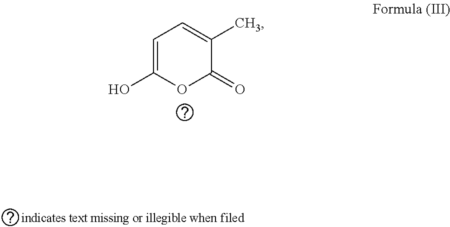
(iv)

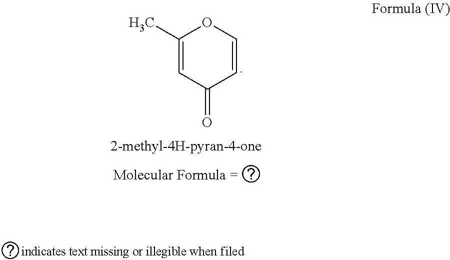

2. The composition as claimed in claim 1, wherein the composition further comprises a pharmaceutically acceptable carrier.

3. The composition as claimed in claim 1, wherein the amount of compound of formula I is in the range of 6 wt % to 18 wt %.

4. The composition as claimed in claim 3, wherein the amount of compound of formula I is 12.60 wt %.

5. The composition as claimed in claim 1, wherein the amount of compound of formula II is in the range of 8 wt % to 22 wt %.

6. The composition as claimed in claim 5, wherein the amount of compound of formula II is 15.41 wt %.

7. The composition as claimed in claim 1, wherein the amount of compound of formula III is in the range of 2 wt % to 10 wt %.

8. The composition as claimed in claim 7, wherein the amount of compound of formula III is 6.48 wt %.

9. The composition as claimed in claim 1, wherein the amount of compound of formula I is in the range of 1 wt % to 10 wt %.

10. The composition as claimed in claim 9, wherein the amount of compound of formula IV is 5.89 wt %.

11. The pharmaceutical composition as claimed in claim 1, wherein the composition further comprises one or more additional therapeutic agent.

12. The pharmaceutical composition as claimed in claim 11, wherein the therapeutic agent is an anticancer agent.

13-18. (canceled)

19. A compound represented by formula I:

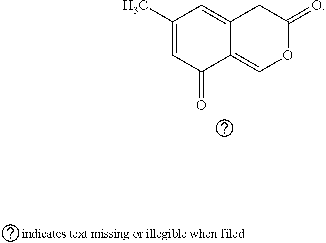

20. A compound represented by formula III:

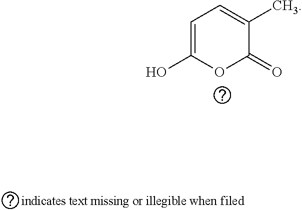

21. (canceled)

22. A method of preparing the pharmaceutical composition as claimed in claim 1 by isolating the compounds of formula I, formula II, formula III, and formula IV, from barley extract, wherein, the method comprising the steps of:
i. performing the chromatography of barley extract by preparative HPLC method;
ii. eluting the peak 2, peak 3, peak 4 and peak 5;
iii. isolating the peaks obtained in step (ii) manually by repetitive injection to obtain fractions;
iv. pooling and concentrating the fractions; and
v. analysing the peaks and determining the structures of compounds of formula I, formula II, formula III, and formula IV;
wherein in step (i), preparative HPLC method comprises mobile phase A and mobile phase B;
wherein in step (ii), elution of peaks 2, 3, 4, and 5, is performed at about 14.3, 15.5, 16.4 and 23.0 minutes respectively.

23. The pharmaceutical composition as claimed in claim 1 wherein the composition is selected from oral, parenteral, nasal, subcutaneous, intradermal, intramuscular, intravenous, intraarticular, intramedullary, intraperitoneal, transmucosal, transdermal, rectal and topical composition.

24. A method of preventing or treating a cancer in a subject, comprising administering to the subject a therapeutically effective amount of the pharmaceutical composition as claimed in claim 1.

25. A method of inducing apoptosis, inducing the expression of pro-apoptotic protein Bax, and inducing the PA RP protein cleavage in a subject, comprising administering to the subject a therapeutically effective amount of the pharmaceutical composition as claimed in claim 1.

26. A method of inhibiting the expression of HER-2, inhibiting anti-apoptotic protein Bcl2, inhibiting the growth of pancreatic cancer cells, degrading the mutation of p53 and normalizing the wild type p53, in a subject, comprising administering to the subject a therapeutically effective amount of the pharmaceutical composition as claimed in claim 1.

27. The method of claim 17, wherein the cancer is selected from the group consisting of: prostate cancer, triple negative breast cancer, invasive cancers, lung cancers, sarcoma, serous carcinoma, pancreatic cancer, and HER2 positive breast cancer.
